SEQUENCE LISTING (25 sequences)

SEQ ID NO: 1 - protein, 10 aa, Artificial
  Peptide linker
LTEEQQEGGG

SEQ ID NO: 2 - protein, 153 aa, Artificial
  Peptide linker
LTEEQQEGGGQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMTEEQQEGGG

SEQ ID NO: 3 - protein, 19 aa, Artificial
  Peptide linker
LAKLKQKTEQLQDRIAGGG

SEQ ID NO: 4 - protein, 65 aa, Artificial
  Peptide linker
LELKTPLGDTTHTCPRCPEPKSCDTPPPCPRCPEPKSCDTPPPCPRCPEPKSCDTPPPCPRCPGG

SEQ ID NO: 5 - protein, 65 aa, Artificial
  Peptide linker
LELKTPLGDTTHTSPRSPEPKSSDTPPPSPRSPEPKSSDTPPPSPRSPEPKSSDTPPPSPRSPGG

SEQ ID NO: 6 - protein, 18 aa, Artificial
  Peptide linker
LEPKSSDKTHTSPPSPGG

SEQ ID NO: 7 - protein, 30 aa, Artificial
  DNA encoding peptide linker
CTTACCGAGGAGCAGCAGGAGGGCGGCGGC

SEQ ID NO: 8 - DNA, 456 bp, Artificial
  DNA encoding peptide linker
cttaccgaggagcagcaggagggcggcggccaggacccctacgtgaaggaggccgagaacctgaagaagtacttcaacgccggccactccgacgtggccgacaacggcaccctgttcctgggcatcctgaagaactggaaggaggagtccgacaggaagatcatgcagtcccagatcgtgtccttctacttcaagctgttcaagaacttcaaggacgaccagtccatccagaagtccgtggagaccatcaaggaggacatgaacgtgaagttcttcaactccaacaagaagaagagggacgacttcgagaagctgaccaactactccgtgaccgacctgaacgtgcagaggaaggccatccacgagctgatccaggtgatggccgagctgtcccccgccgccaagaccggcaagaggaagaggtcccagatgaccgaggagcagcaggagggcggc

SEQ ID NO: 9 - DNA, 57 bp, Artificial
  DNA encoding peptide linker
cttgctaaattaaaacaaaaaactgaacaattacaagatcgtattgctggtggcggc

SEQ ID NO: 10 - DNA, 195 bp, Artificial
  DNA encoding peptide linker
cttgagctcaaaaccccacttggtgacacaactcacacatgcccacggtgcccagagcccaaatcttgtgacacacctcccccgtgcccaaggtgcccagagcccaaatcttgtgacacacctcccccgtgcccaaggtgcccagagcccaaatcttgtgacacacctcccccgtgcccaaggtgcccaggcggc

SEQ ID NO: 11 - DNA, 159 bp, Artificial
  DNA encoding peptide linker
cttgagctcaaaaccccacttggtgacacaactcacacatccccacggtccccagagcccaaatcttctgacacacctcccccgtccccaaggtccccagagcccaaatcttctgacacacctcccccgtccccaaggtccccaaggtccccaggcggc

SEQ ID NO: 12 - DNA, 54 bp, Artificial
  DNA encoding peptide linker
cttgagcccaaatcttccgacaaaactcacacatctccaccgtccccaggcggc

SEQ ID NO: 13 - protein, 133 aa, Homo sapiens
DPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQM

SEQ ID NO: 14 - protein, 141 aa, Artificial
  Anti-CD138 heavy chain with leader sequence
MGWSYIILFLVATATGVHSQVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 15 - protein, 127 aa, Artificial
  Anti-CD138 light chain with leader sequence
MKSQTQVFIFLLLCVSGAHGDIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 16 - protein, 469 aa, Artificial
  Anti-HER2 heavy chain with leader sequence
MECSWVMLFLLSVTAGVHSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 233 aa, Artificial
  Anti-HER2 light chain with leader sequence
MEWSCVMLFLLSVTAGVHSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFEPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 470 aa, Artificial
  Anti-CD20 heavy chain with leader sequence
MYLGLNCVIIVFLLKGVQSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 232 aa, Artificial
  Anti-CD20 light chain with leader sequence
MKLPVRLLVLMFWIPASSSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 463 aa, Artificial
  Anti-endoplasmin heavy chain with leader sequence
MYLGLNCVIIVFLLKGVQSQVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARAHFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 234 aa, Artificial
  Anti-endoplasmin light chain with leader sequence
MEAPAQLLFLLLLWLPDTTGEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 465 aa, Artificial
  Anti-CD33 heavy chain with leader sequence
MEWSWVFLFFLSVTTGVHSQVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 238 aa, Artificial
  Anti-CD33 light chain with leader sequence
MSVPTQVLGLLLLWLTDARCDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 152 aa, Artificial
  Anti-CD276 heavy chain with leader sequence
MNFGFRLIFLALILKGVQCEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSSAKTTAPSVYPLAPGSL

SEQ ID NO: 25 - protein, 157 aa, Artificial
  Anti-CD276 light chain with leader sequence
MKSQSQVFVFVFLWLSGVDGDIVMTQFAGVDGDIVMTQSHKFMSTSVGDRVSITCKASQDVSTTVAWYQQKPGQSPKLLIYSASYRYTGVPDRFTGSGSGTDFTFTISSVQAEDLAVYYCQQHYSTPPTFGGGTKLEIKRADAAPTVSIFPPSSKLG

(19) United States
(12) Patent Application Publication
(10) Pub. No.: US 2022/0177598 A1
(43) Pub. Date: Jun. 9, 2022

(54) TARGETED DELIVERY OF IFN GAMMA USING ANTIBODY FUSION PROTEINS

(71) Applicant: The Regents of the University of California, Oakland, CA (US)

(72) Inventors: Kham M. Trinh, Alhambra, CA (US); John Matthew Timmerman, Los Angeles, CA (US); Sherie Leaver Morrison, Los Angeles, CA (US)

(21) Appl. No.: 17/310,279

(22) PCT Filed: Jan. 29, 2020
(86) PCT No.: PCT/US2020/015727

§ 371 Date: Jul. 27, 2021

Related U.S. Application Data
(60) Provisional application No. 62/798,994, filed on Jan. 30, 2019.

(51) Int. Cl. C07K 16/30; C07K 16/28; C07K 14/57; A61P 35/00

(52) U.S. Cl. CPC C07K 16/30; C07K 16/2896; C07K 16/2887; C07K 14/57; C07K 2317/734; C07K 2317/22; C07K 2317/53; C07K 2317/732; A61P 35/00

(57) ABSTRACT

In various embodiments targeted interferon constructs are provided. In certain embodiments the constructs comprise a full-length immunoglobulin or a camelid antibody attached to an interferon gamma (IFNγ) where said immunoglobulin or camelid antibody is an antibody that binds to a tumor associated antigen; a first interferon gamma (IFNγ) is attached to a first constant heavy region 3 (CH₃) of the immunoglobulin or camelid antibody by a first proteolysis resistant peptide linker; a second interferon gamma is attached to a second constant heavy region 3 (CH₃) of the immunoglobulin or camelid antibody by a second proteolysis resistant peptide linker; and the first proteolysis resistant linker and the second proteolysis linker have a length and flexibility that permits said first interferon gamma and said second interferon gamma to dimerize.

### CROSS-REFERENCE TO RELATED APPLICATIONS

This application claims benefit of and priority to U.S. Ser. No. 62/798,994, filed on Jan. 30, 2019, which is incorporated herein by reference in its entirety for all purposes.

### STATEMENT OF GOVERNMENTAL SUPPORT

This invention was made with government support under Grant No. R01 CA200910 awarded by the National Institutes of Health. The Government has certain rights in this invention.

### BACKGROUND

Interferon-gamma (IFN-γ) is a pleiotropic cytokine secreted by Natural Killer (NK) cells and many T cells including NKT. It is the sole representative of Type II IFN and is often termed immune interferon. IFN-γ has pleiotropic activities including: directly inhibiting cell growth, inducing an antiviral state in cells, upregulating cell surface markers such as Class I and II MHC, and regulating the activity of macrophages, NK cells, neutrophils, B-cells, and T-cells. As such, IFN-γ has broad appeal for use as a cancer therapeutic.

IFN-γ functions as a dimeric cytokine that binds to a heterodimeric receptor consisting of the IFN-γ Receptor Chain 1 (IFNGR1), which provides binding affinity, and Receptor Chain 2 (IFNGR2), which is involved in signal transduction. Recent evidence has suggested that the receptor actually has a tetrameric structure consisting of two IFNGR2 molecules and two IFNGR1 molecules. Binding of IFN-γ leads to a conformational change and subsequent signal transduction (Krause and Pestka (2007) Cytokine Growth Factor Reviews 18: 473).

The human IFNγ gene encodes a polypeptide of 143 amino acid residues. Natural forms of human IFNγ are modified to contain N-linked glycosylation at two positions, and a heterogeneous C terminus. Mouse IFNγ contains only 2 potential sites of N-glycosylation. However, glycosylation is not necessary for activity as recombinant IFNγ produced in E. coli is active. Human IFNγ has a heterogeneous carboxy-terminus and studies have shown that proteins missing 9 of the c-terminal amino acids have improved anti-viral activity. The crystal structure of human IFNγ revealed an alpha-helical homodimer composed of two peptide chains. Each domain consists of six tightly associated α-helices of which four are donated from one peptide chain and the last two from the other to form intertwined or domain-swapped dimers. This dimer binds two copies of IFNGR1 in solution and the IFN-γ dimer structure is important for full activity.

### SUMMARY

Various embodiments contemplated herein may include, but need not be limited to, one or more of the following:

Embodiment 1: A chimeric construct comprising a full-length immunoglobulin or a camelid antibody attached to an interferon gamma (IFNγ) wherein:
said immunoglobulin or camelid antibody is an antibody that binds to a tumor associated antigen;
a first interferon gamma (IFNγ) is attached to a first constant heavy region 3 (CH₃) of said immunoglobulin or camelid antibody by a first proteolysis resistant peptide linker;
a second interferon gamma is attached to a second constant heavy region 3 (CH₃) of said immunoglobulin or camelid antibody by a second proteolysis resistant peptide linker; and
said first proteolysis resistant linker and said second proteolysis linker have a length and flexibility that permits said first interferon gamma and said second interferon gamma to dimerize.

Embodiment 2: The construct of embodiment 1, wherein said first proteolysis resistant peptide linker and said second proteolysis peptide linker comprise amino acid sequences independently selected from the amino acid sequences of the peptide linkers shown in Table 1.

Embodiment 3: The construct of embodiment 2, wherein said first proteolysis resisting peptide linker and/or said second proteolysis resisting peptide linker comprise the amino acid sequence of the Landar linker.

Embodiment 4: The construct of embodiment 2, wherein said first proteolysis resisting peptide linker and/or said second proteolysis resisting peptide linker comprise the amino acid sequence of the Double landar linker.

Embodiment 5: The construct of embodiment 2, wherein said first proteolysis resisting peptide linker and/or said second proteolysis resisting peptide linker comprise the amino acid sequence of the 1qo0E_1 linker.

Embodiment 6: The construct of embodiment 2, wherein said first proteolysis resisting peptide linker and/or said second proteolysis resisting peptide linker comprise the amino acid sequence of the IgG3 hinge linker.

Embodiment 7: The construct of embodiment 2, wherein said first proteolysis resisting peptide linker and/or said second proteolysis resisting peptide linker comprise the amino acid sequence of the IgG3 hinge delta cys linker.

Embodiment 8: The construct of embodiment 2, wherein said first proteolysis resisting peptide linker and/or said second proteolysis resisting peptide linker comprise the amino acid sequence of the IgG1 hinge delta cys linker.

Embodiment 9: The construct according to any one of embodiments 1-8, wherein said interferon gamma comprises a murine interferon gamma, or a truncated and/or mutated murine interferon gamma.

Embodiment 10: The construct of embodiment 9, wherein said interferon gamma comprises a full-length murine interferon gamma.

Embodiment 11: The construct according to any one of embodiments 9-10, wherein said murine interferon gamma is not glycosylated.

Embodiment 12: The construct according to any one of embodiments 9-10 wherein said murine interferon gamma is glycosylated at Asn 38 and/or at ASN 90.

Embodiment 13: The construct according to any one of embodiments 1-8, wherein said interferon gamma comprises a human interferon gamma or a truncated and/or mutated human interferon gamma.

Embodiment 14: The construct of embodiment 13, wherein said human interferon gamma is not glycosylated.

Embodiment 15: The construct of embodiment 13, wherein said human interferon gamma comprises N-linked glycosylation at Asn-25 and/or at Asn-97.

Embodiment 16: The construct according to any one of embodiments 13-15, wherein said human interferon gamma comprises a full-length human interferon.

Embodiment 17: The construct according to any one of embodiments 13-15, wherein said human interferon gamma comprises a huIFNγ C-terminally truncated with 1-15 amino acid residues, e.g. with 1, 2, 3, 4, 5, 6, 7, 8, 9, 10, 11, 12, 13, 14, or 15 amino acid residues.

Embodiment 18: The construct according to any one of embodiments 13-15, and 17, wherein said human interferon gamma comprises a human interferon gamma N-terminally truncated with 1, 2, or 3 amino acid residues.

Embodiment 19: The construct according to any one of embodiments 13-15, wherein said human interferon gamma comprise a human interferon gamma with an N-terminal addition CYC.

Embodiment 20: The construct according to any one of embodiments 13-19, wherein said human interferon gamma comprises a cysteine substitutions at one or more of Glu8, Ser70, Ala18, His112, Lys81, Leu121, Gln49, and Leu96 (relative to the amino acid sequence of SEQ ID NO: 13).

Embodiment 21: The construct of embodiment 20, wherein said human interferon gamma comprises at least one pair of amino acids from predetermined amino acid pairs exchanged for cysteine, wherein said four amino acid pairs are Glu8 and Ser70, Ala18 and His 112, Lys81 and Leu121, and Gln-49 and Leu96.

Embodiment 22: The construct according to any one of embodiments 1-21, wherein said antibody or camelid antibody preferentially or specifically binds to a tumor associated antigen (TAA) selected from the group consisting of CD138, CSPG4, α-fetoprotein, 5 alpha reductase, 5T4 (or TPBG, trophoblast glycoprotein), AM-1, APC, APRIL, B7 family members, BAGE, Bc12, bcr-abl (b3a2), CA-125, CASP-8/FLICE, Cathepsins, CD1, CD115, CD123, CD13, CD14, CD15, CD19, CD2, CD20, CD200, CD203c, CD21, CD23, CD22, CD38, CD25, CD276, CD3, CD30, CD303, CD33, CD34, CD35, CD37, CD38, CD44, CD45, CD46, CDS, CD52, CD55, CD56, CD59 (791Tgp72), CD7, CD70, CD74, CD79, CDC127, CDK4, CEA, CLL-1, c-MET (or HGFR), c-myc, Cox-2, Cripto, DCC, DcR3, DLL3, E6/E7, EGFR, EMBP, Ena78, endoplasmin, EPCAM, EphA2, EphB3, ETBR, FcRL5, FGF8b and FGF8a, FLK-1/KDR, FOLR1, G250, GAGE-Family, gastrin 17, gastrin-releasing hormone (bombesin), GD2/GD3/GM2, glutathione 5-transferase, glycosphingolipid GD2, GnRH, GnTV, gp100/Pme117, gp-100-in4, gp15, gp75/TRP-1, GPNMB, hCG, Heparanase, Her2/neu, Her3, Her4, HLA-DR, HM 1.24, HMB 45, HMTV, HMW-MAA, Hsp70, hTERT (telomerase), IGFR1, IL-13R, iNOS, integrin, Ki 67, KIAA0205, K-ras, H-ras, N-ras, KSA (CO17-1A), LDLR-FUT, Leu-M1, Lewis A like carbohydrate, Lewis Y, LIV1, MAGE1, MAGES, Mammaglobin, MAP17, Melan-A/, MART-1, mesothelin, MIC A/B, MN, Mox1, MMP2, MMP3, MMP7, MMP9, MUC16, MUC-1, MUC-2, MUC-3, MUC-4, MUC-16, MUM-1, NaPi2b, Nectin-4, NY-ESO-1, Osteonectin, p15, p16INK4, P170/MDR1, p53, p97/melanotransferrin, PAI-1, PDGF, plasminogen (uPA), PMSA, PRAME, Probasin, Progenipoietin, PSA (phosphatidyl serine antigen), PSM, RAGE-1, Rb, RCAS1, SART-1, SE10, SIRP.alpha., SLAM family members, SLC44A4, SSX gene, family, STAT3, STEAP-1, STn (mucin assoc.), TAG-72, TF (or tissue factor), TGF-α, TGF-β, Thymosin β15, TNF superfamily members, TPA, TPI, TRP-2, Tyrosinase, VEGF, VLA, ZAG, and β-catenin.

Embodiment 23: The construct of embodiment 22, wherein said antibody or camelid antibody preferentially or specifically binds to CSPG4.

Embodiment 24: The construct of embodiment 23, wherein said antibody comprises the CDRs of an antibody selected from the group consisting of 9.2.27, VF1-TP34, VF1-TP34, VF1-TP41.2, TP61.5, 149.53, 149.53, 225.28, 225.28s, 763.74, and scFv-FcC21.

Embodiment 25: The construct of embodiment 22, wherein said antibody or camelid antibody preferentially or specifically binds to CD138.

Embodiment 26: The construct of embodiment 25, wherein said antibody comprises the CDRs of an antibody comprises an antibody selected from the group consisting of nBT062, B-B4, BC/B-B4, B-B2, DL-101, 1 D4, MI15, 1.BB.210, 2Q1484, 5F7, 104-9, and 281-2.

Embodiment 27: The construct of embodiment 22, wherein said antibody or camelid antibody preferentially or specifically binds to a member of the EGF receptor family.

Embodiment 28: The construct of embodiment 27, wherein said antibody comprises the CDRs of an antibody comprises an antibody selected from the group consisting of C6.5, C6ML3-9, C6MH3-B1, C6-B1D2, F5, HER3.A5, HER3.F4, HER3.H1, HER3.H3, HER3.E12, HER3.B12, EGFR.E12, EGFR.C10, EGFR.B11, EGFR.E8, HER4.B4, HER4.G4, HER4.F4, HER4.A8, HER4.B6, HER4.D4, HER4.D7, HER4.D11, HER4.D12, HER4.E3, HER4.E7, HER4.F8 and HER4.C7.

Embodiment 29: The construct of embodiment 22, wherein said antibody or camelid antibody preferentially or specifically binds to CD20.

Embodiment 30: The construct of embodiment 29, wherein said antibody comprises the CDRs of an antibody comprises an antibody selected from the group consisting of rituximab, Ibritumomab tiuxetan, and tositumomab.

Embodiment 31: The construct of embodiment 22, wherein said antibody or camelid antibody preferentially or specifically binds to endoplasmin.

Embodiment 32: The construct of embodiment 22, wherein said antibody or camelid antibody preferentially or specifically binds to CD33.

Embodiment 33: The construct of embodiment 22, wherein said antibody or camelid antibody preferentially or specifically binds to CD276.

Embodiment 34: The construct according to any one of embodiments 1-33, wherein said antibody or camelid antibody is a full-length immunoglobulin.

Embodiment 35: The construct of embodiment 34, wherein said antibody is a human antibody.

Embodiment 36: The construct of embodiment 34, wherein said antibody is a humanized or chimeric antibody.

Embodiment 37: The construct according to any one of embodiments 1-33, wherein said antibody or camelid antibody is a camelid antibody.

Embodiment 38: A pharmaceutical formulation comprising:

a chimeric construct according to any one of embodiments 1-37; and

a pharmaceutically acceptable carrier.

Embodiment 39: The pharmaceutical formulation of embodiment 38, wherein said formulation is a unit dosage formulation.

Embodiment 40: The formulation according to any one of embodiments 38-39, wherein said formulation is formulated for administration via a route selected from the group consisting of oral administration, nasal administration, rectal administration, intraperitoneal injection, intravascular injection, subcutaneous injection, transcutaneous administration, and intramuscular injection.

Embodiment 41: A method of inhibiting growth and/or proliferation of a cell that expresses or overexpresses CD138, said method comprising contacting said cell with a chimeric construct according to any of embodiments 1-36, or a formulation according to any one of embodiments 37-39 in an amount sufficient to inhibit growth or proliferation of said cell.

Embodiment 42: The method of embodiment 41, wherein said cell is a cancer cell.

Embodiment 43: The method of embodiment 42, wherein said cancer cell is a metastatic cell.

Embodiment 44: The method of embodiment 42, wherein said cancer cell is in a solid tumor.

Embodiment 45: The method of embodiment 42, wherein said cancer cell is cell produced by a cancer selected from the group consisting of multiple myeloma, ovarian carcinoma, cervical cancer, endometrial cancer, kidney carcinoma, gall bladder carcinoma, transitional cell bladder carcinoma, gastric cancer, prostate adenocarcinoma, breast cancer, prostate cancer, lung cancer, colon carcinoma, Hodgkin's and non-Hodgkin's lymphoma, chronic lymphocytic leukemia (CLL), acute lymphoblastic leukemia (ALL), acute myeloblastic leukemia (AML), a solid tissue sarcoma, colon carcinoma, non-small cell lung carcinoma, squamous cell lung carcinoma, colorectal carcinoma, hepato-carcinoma, pancreatic cancer, and head and neck carcinoma.

Embodiment 46: The method of embodiment 42, wherein said cancer cell is a cell of a multiple myeloma.

Embodiment 47: The method according to any one of embodiments 41-46, wherein said method comprises inhibiting, delaying and/or preventing the growth of a tumor and/or spread of malignant tumor cells.

Embodiment 48: The method according to any one of embodiments 41-47, wherein said contacting comprises systemically administering said construct or formulation to a mammal.

Embodiment 49: The method according to any one of embodiments 41-47, wherein said contacting comprises administering said construct or formulation directly into a tumor site.

Embodiment 50: The method according to any one of embodiments 41-47, wherein said contacting comprises administering said construct or formulation via a route selected from the group consisting of oral administration, intravenous administration, intramuscular administration, direct tumor administration, inhalation, rectal administration, vaginal administration, transdermal administration, and subcutaneous depot administration.

Embodiment 51: The method according to any one of embodiments 41-47, wherein said contacting comprises administering said construct or formulation intravenously.

Embodiment 52: The method according to any one of embodiments 41-51, wherein said cell is a cell in a human.

Embodiment 53: The method according to any one of embodiments 41-51, wherein said cell is a cell in a non-human mammal.

Embodiment 54: The method of embodiment 41, wherein said cancer cell is a cell produced by a multiple myeloma.

Embodiment 55: The method according to any one of embodiments 41-54, wherein said method comprises co-administration of said chimeric construct with bortezomib.

Embodiment 55: The method according to any one of embodiments 41-55, wherein said method comprises co-administration of said chimeric construct with ibrutinib.

Embodiment 57: The method according to any one of embodiments 55-55, wherein said co-administration provides a synergistic effect.

### Definitions

The terms “targeted interferon” as used herein refers to an interferon attached to a “targeting moiety” (e.g., an antibody) that binds to a molecule disposed, for example, on the surface of a cell (e.g., a cancer cell). One illustrative targeted interferon comprises a chimeric moiety comprising an antibody or a camelid antibody attached to two interferon gamma molecules as described herein.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide. Preferred “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to peptide mimetics such as amino acids joined by an ether as opposed to an amide bond.

An “antibody”, as used herein, refers to a protein consisting of one or more polypeptides substantially encoded by immunoglobulin genes or fragments of immunoglobulin genes. In certain embodiments, the immunoglobulin genes are human immunoglobulin genes. Recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively.

A typical (native) immunoglobulin (antibody) (full-length antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these regions of the light and heavy chains respectively. It is noted that immunoglobulins IgA and IgM contain multiple copies of the four chain structure.

The phrase “inhibition of growth and/or proliferation” of a cancer cell refers to decrease in the growth rate and/or proliferation rate of a cancer cell. In certain embodiments this includes death of a cancer cell (e.g. via apoptosis). In certain embodiments this term also refers to inhibiting the growth and/or proliferation of a solid tumor and/or inducing tumor size reduction or elimination of the tumor.

The terms “tumor associated antigen”, “TAA”, and “cancer marker” are used interchangeably to refer to biomolecules such as proteins, carbohydrates, glycoproteins, and the like that are exclusively or preferentially or differentially expressed on a cancer cell and/or are found in association with a cancer cell and thereby provide targets preferential or specific to the cancer. In various embodiments the preferential expression can be preferential expression as compared to any other cell in the organism, or preferential expression within a particular area of the organism (e.g. within a particular organ or tissue).

The terms “subject,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the subject can be a human (e g, adult male, adult female, adolescent male, adolescent female, male child, female child) under the care of a physician or other health worker in a hospital, psychiatric care facility, as an outpatient, or other clinical context. In certain embodiments, the subject may not be under the care or prescription of a physician or other health worker.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like. Where administration is described herein, “causing to be administered” is also contemplated.

The term “exhibiting IFN gamma activity” is intended to indicate that the polypeptide has one or more of the functions of native IFNγ, in particular huIFNγ or rhuIFNγ. Such functions include, inter alia, the capability to bind to an IFNγ receptor and cause transduction of the signal transduced upon huIFNγ-binding of its receptor as determined in vitro or in vivo (i.e., in vitro or in vivo bioactivity). The IFNγ receptor has been described by Aguet et al. (1988) Cell 55: 273-280) and Calderon et al. (1988) Proc. Natl. Acad. Sci. USA, 85:4837-4841. The “IFNγ polypeptide” is a polypeptide exhibiting IFNγ activity and is used herein about the polypeptide in monomer or dimeric form, as appropriate. For instance, when specific substitutions are indicated these are normally indicated relative to the IFNγ polypeptide monomer. When reference is made to the IFNγ as part of a conjugate this is normally in dimeric form (and thus, e.g., comprises two IFNγ polypeptide monomers modified as described). The dimeric form of the IFNγ polypeptides may be provided by the normal association of two monomers or be in the form of a single chain dimeric IFNγ polypeptide. The IFNγ polypeptide described herein may have an in vivo or in vitro bioactivity of the same magnitude as huJFNγ or rhuIFNγ or lower or higher, e.g. an in vivo or in vitro bioactivity of >100% (e.g., 125% or greater, or 150% or greater, or 200% or greater, or 300% or greater, or 400% or greater, or 500% or greater, or 1000% (10-fold) or greater, and so forth), 1-100% of that of huIFNγ or rhuIFNγ, as measured under the same conditions, e.g. 1-25% or 1-50% or 25-100% or 50-100% of that of huIFNγ or rhuIFNγ.

### BRIEF DESCRIPTION OF THE DRAWINGS

FIG. 1. Inhibition of proliferation by anti-CSPG4 fusions with IFNγ using different linkers. OVCAR and T98MG cells were treated for 3 days and A375 cells were treated with 4 days with the indicated fusion proteins at different concentrations and the metabolic activity of the remaining cells determined using the MTS assay.

FIG. 2. Inhibition of proliferation by anti-CSPG4 fusions with IFNγ using different linkers. Cells were treated for 6 days with the indicated fusion proteins at different concentrations and the metabolic activity of the remaining cells determined using the MTS assay.

FIG. 3. Ovarian cancer cell lines were stained with the indicated antibodies and analyzed by flow cytometry.

FIG. 4. Ovarian cancer cell lines treated with targeted anti-CD138, anti-CD138-IFNα2, anti-CD138-IFNα14, anti-CD138-IFNα2^{YNS}, anti-CD138IFNγ, or untargeted anti-CD20-IFNα2^{YNS}, or anti-CD20-IFNγ. Cells were treated for 6 days and proliferation analyzed by MTS.

FIG. 5. Inhibition of proliferation of ovarian cell lines following treatment with hIFNγ, targeted anti-CD138IFNγ, or untargeted anti-CD20-IFNγ for 6 days.

FIG. 6. Inhibition of proliferation of the glioblastoma T98MG following treatment with targeted unfused anti-CD138, anti-CD138-IFNα2, anti-CD138-IFNα14, anti-CD138-IFNα2^{YNS}, or anti-CD138IFNγ or untargeted anti-CD20-IFNγ for 6 days. Metabolic activity of surviving cells was determined using the MTS assay.

FIG. 7. Inhibition of proliferation of the multiple myeloma cell lines OCI-My5, H929, MM1-144, ANBL-6, 8226Dox40 and U266 following treatment with targeted anti-CD138IFNγ or untargeted anti-CD20-IFNγ for 3 days and metabolic activity of surviving cells was determined using the MTS assay.

FIG. 8. B16 mouse myeloma cells were incubated with either 1.5 nM mouse IFNγ (mIFNγ) or 0.75 nM anti-CD20-mIFNγ for 24 hours, stained with PE-labeled mouse anti-H2-K^{b} and analyzed by flow-cytometry. Controls included cells that were not treated with INFγ (Untreated) but were stained with anti-H2-K^{b} and cells treated with IFNγ but stained with a mouse antibody of irrelevant specificity.

FIG. 9. Inhibition of proliferation of the murine melanoma B16 following treatment with untargeted mIFNγ or anti-CD20-mIFNγ for 4 days. Metabolic activity of surviving cells was determined using the MTS assay.

FIG. 10. Inhibition of proliferation of the murine melanoma B16 expressing human CD20 following treatment with untargeted mIFNα, mIFNβ or mIFNγ or targeted anti-CD20-mIFNα, anti-CD20-mIFNβ or anti-CD20-mIFNγ for 4 days. Metabolic activity of surviving cells was determined using the MTS assay.

FIG. 11. Effect of treatment on in vivo tumor growth. C57/BL6 mice injected subcutaneously with B16huCD20 cells were treated on days 5, 6 and 7 with either PBS or 100 μg of anti-hCD20-mIFNγ and monitored for tumor growth. Mice were sacrificed when tumors exceeded 1.5 cm in diameter per institutional guidelines.

FIG. 12. Rabbit complement lysis of 38C13-huCD20.

FIG. 13. ADCC of 38C13-huCD20.

FIG. 14. Expression of MHCI, ICAMI, PDL-L1, CD80 and FAS in 38C13-huCD20 following 24 hours treatment with 10 nM mIFNγ, anti-CD20 or anti-CD20-mIgG2a-mIFNγ.

FIG. 15. Inhibition of proliferation of the murine lymphoma 38C13 following treatment with untargeted mIFNγ or anti-CD20-mIFNγ for 4 days. Metabolic activity of surviving cells was determined using the MTS assay.

FIG. 16. Inhibition of proliferation of the murine lymphoma 38C13huCD20 following treatment with the indicated proteins for 4 days. Metabolic activity of surviving cells was determined using the MTS assay.

FIG. 17. Three independent experiments showing effect of treatment on tumor growth. C3H/HeJ mice injected subcutaneously with 38C13huCD20 cells were treated on days 5, 6 and 7 with either PBS or 100 μg of anti-hCD20-mIFNγ and monitored for tumor growth. Mice were sacrificed when tumors exceeded 1.5 cm in diameter per institutional guidelines.

FIG. 18, panels A-C: Anti-hCD20mIgG2a-mIFNγ was more effective in inhibiting tumor growth in vivo than anti-hCD20mIgG2a. C3H/HeJ mice injected subcutaneously with 5000 (panels A and B) or 4000 (panel C) 38C13huCD20 cells were either treated on days 5, 6 and 7 (panel A) or on days 4, 5, 6, and 8 (panels B and C) with either PBS or 100 μg of anti-hCD20-mIFNγ or an equivalent molar concentration of anti-hCD20mIgG2a and monitored for tumor growth. Mice were sacrificed when tumors exceeded 1.5 cm in diameter per institutional guidelines.

FIG. 19. Higher doses of anti-hCD20mIgG2a-mIFN□ are more effective in inhibiting tumor growth in vivo. C3H/HeJ mice injected subcutaneously with 38C13huCD20 cells were either treated on days 5, 6 and 7 either PBS, 100 μg of anti-hCD20-mIFNγ. 50 μg of anti-hCD20-mIFNγ or 25 μg of anti-hCD20-mIFNγ and monitored for tumor growth. Mice were sacrificed when tumors exceeded 1.5 cm in diameter per institutional guidelines.

FIG. 20, panels A-F: To evaluate the ability of the fusion proteins to induce apoptosis in MWCL-1, cells were treated with 12.5 nM of the indicated proteins for 5 days and apoptosis evaluated by flow-cytometry following staining with Annexin V and PI.

FIG. 21, panels A-F: To determine the ability of the proteins to inhibit proliferation, MWCL-1 cells were incubated with 12.5 nM protein for 72 hours, then pulsed with ³H-thymidine for 8 hours and proliferation measured by determining the amount of ³H-thymidine incorporated into DNA.

FIG. 22, panels A-E, illustrates STAT activation (pSTAT1, total STAT1, pSTAT3 and total STATS) following treatment with the various interferon constructs. Panel A) Comparison of STAT activation by anti-CD138, anti-CD20, anti-CD138-IFNα2, anti-CD138-IFNγ, anti-CD138-IFNα14, anti-CD20-IFNα14, anti-CD138-IFNα2^{YNS}, anti-CD20-IFNα2^{YNS}, and anti-CD20-IFNγ. Panel B) Comparison of STAT activation by anti-CD138-IFNα2+anti-CD20-IFNα2, anti-CD138-IFNα14+anti-CD20-IFNα14, anti-CD138-IFNα2^{YNS}+anti-CD20-IFNα2^{YNS}. Panel C) Comparison of STAT activation by anti-CD138-IFNγ+anti-CD138, anti-CD138-IFNγ+anti-CD138-IFNα2, anti-CD138-IFNγ+anti-CD138-IFNα14, anti-CD138-IFNγ+anti-CD138-IFNα2^{YNS}, anti-CD138-IFNγ+anti-CD20, anti-CD138-IFNγ+anti-CD20-IFNα2, anti-CD138γ+anti-CD20-IFNα14, anti-CD138γ+anti-CD20-IFNα2^{YNS}. Panel D) Comparison of STAT activation by anti-CD20-IFNγ+anti-CD20, anti-CD20-IFNγ+anti-CD138-IFNα2, anti-CD20γ+anti-CD138-IFNα14, anti-CD20-IFNγ+anti-CD138-IFNα2^{YNS}, anti-CD20-IFNγ+anti-CD20, anti-CD20-IFNγ+anti-CD20-IFNα2, anti-CD20-IFNγ+anti-CD20-IFNα14, anti-CD20-IFNγ+anti-CD20-IFNα2^{YNS}. Panel E) Comparison of STAT activation by anti-CD138IFNα2, anti-CD138-IFNα2+anti-CD138-IFNγ, anti-CD138-IFNγ; anti-CD20-IFNα2+anti-cd138-IFNγ. Anti-CD20-IFNα2, anti-CD138-IFNα2, anti-CD138-IFNα2+anti-CD20-IFNγ, anti-cd20-IFNγ, anti-CD20IFNα2+anti-CD20-IFNγ, and anti-CD20-IFNα2.

FIG. 23, panels A-C, illustrates anti-proliferative activity of IFN-γ fusion proteins in combination with bortezomib or ibrutinib. Panel A) First experiment. Panel B) Second experiment. Panel C) Combination of experiment 1 and experiment 2.

FIG. 24, panels A-C, illustrates anti-proliferative activity of IFN-γ fusion proteins in combination with bortezomib or ibrutinib. Panel A) Fusion protein concentrations at 25,000 pM, 5,000 pM, and 1,000 pM. Panel B) Bortezomib antiproliferative activity. Panel C) Ibrutinib antiproliferative activity.

### DETAILED DESCRIPTION

In view of the requirement for dimer formation for optimal interferon gamma (IFNγ) activity, we examined the use of a number of different linkers for their ability to allow functional dimer formation by two IFNγ moieties joined to the carboxy termini of the IgG heavy chains of a full-length antibody. One challenge was to make sure that linkers are long enough so that different domains do not impair each other and inhibit biological activity while permitting the formation of active interferon gamma dimers. Additionally, linkers were selected to resist or avoid proteolytic cleavage (proteolysis resistant linkers). Linkers that we examined are illustrated in Table 1.

TABLE 1
Linker sequences.

| Linker | | Nucleotide |
|---|---|---|
| Name | Amino Add Sequence | Sequence |
| Landar | L T E E Q Q E G G G | CTTACCGAGGAGCAG |
| | (SEQ ID NO: 1) | CAGGAGGGCGGCGGC |
| | | (SEQ ID NO: 7) |
| Double | L T E E Q Q E G G G- | CTTACCGAGGAGCAG |
| Landar | hIFNγ-T E E Q Q | CAGGAGGGCGGCGGC- |
| | E G G G | hIFNγ-ACCGAG |
| | (SEQ ID NO: 2) | GAGCAGCAGGAGGGC |
| | | GGCGGC |
| | | (SEQ ID NO: 8) |
| 1qo0E_1 | L A K L K Q K T E Q | CTTGCTAAATTAAAAC |
| | L Q D R I A G G G | AAAAAACTGAACAATT |
| | (SEQ ID NO: 3) | ACAAGATCGTATTGCT |
| | | GGTGGCGGC |
| | | (SEQ ID NO: 9) |
| IgG3 hinge | L E L K T P L G D T | CTTGAGCTCAAAACCCC |
| | T H T C P R C P E P | ACTTGGTGACACAACTC |
| | K S C D T P P P C P | ACACATGCCCACGGTGC |
| | R C P E P K S C D T | CCAGAGCCCAAATCTTG |
| | P P P C P R C P E P | TGACACACCTCCCCCGT |
| | K S C D T P P P C P | GCCCAAGGTGCCCAGAG |
| | R C P G G | CCCAAATCTTGTGACAC |
| | (SEQ ID NO: 4) | ACCTCCCCCGTGCCCAA |
| | | GGTGCCCAGAGCCCAAA |
| | | TCTTGTGACACACCTCC |
| | | CCCGTGCCCAACGTGCC |
| | | CAGGCGGC |
| | | (SEQ ID NO: 10) |
| IgG3 | L E L K T P L G D T | CTTGAGCTCAAAACCCC |
| HingeΔcys | T H T S P R S P E P | ACTTGGTGACACAACTC |
| | K S S D T P P P S P | ACACATCCCCACGGTCC |
| | R S P E P K S S D T | CCAGAGCCCAAATCTTC |
| | P P P S P R S P E P | TGACACACCTCCCCCGT |
| | K S S D T P P P S P | CCCCAAGGTCCCGAGAG |
| | R S P G G | CCCAAATCTTCTGACAC |
| | {SEQ ID NO: 5) | ACCTCCCCCGTCCCCAA |
| | | GGTCCCCAGAGCCCAAA |
| | | TCTTCTGACACACCTCC |
| | | CCCGTCCCCAAGGTCCC |
| | | CAGGCGGC |
| | | (SEQ ID NO: 11) |
| IgG1 | L E P K S S D K T H | CTTGAGCCCAAATCTTC |
| HingeΔcys | T S P P S P G G | CGACAAAACTCACACAT |
| | (SEQ ID NO: 6) | CTCCACCGTCCCCAGGC |
| | | GGC |
| | | (SEQ ID NO: 12) |

The linkers have different properties. IgG1 delta cys and 1qo0E_1 are similar in length (18 and 19 aa, respectively) but have different conformations (coil and alpha helix, respectively). IgG3 and IgG3 delta cys are of the same length but there are no disulfide bonds in the latter. The Landar linker has a relatively short 10 aa sequence.

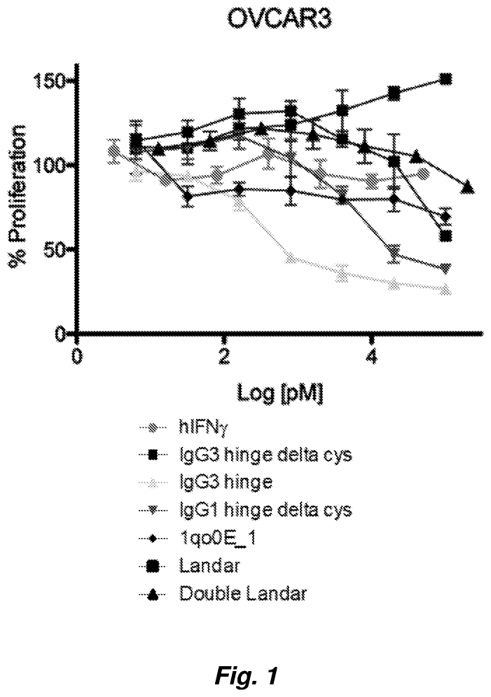

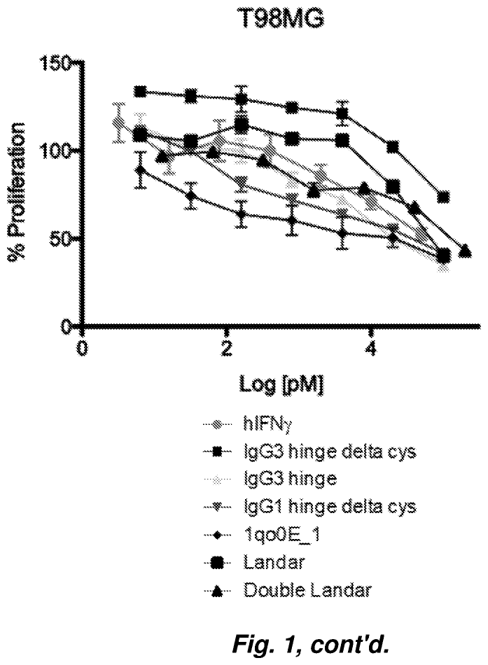

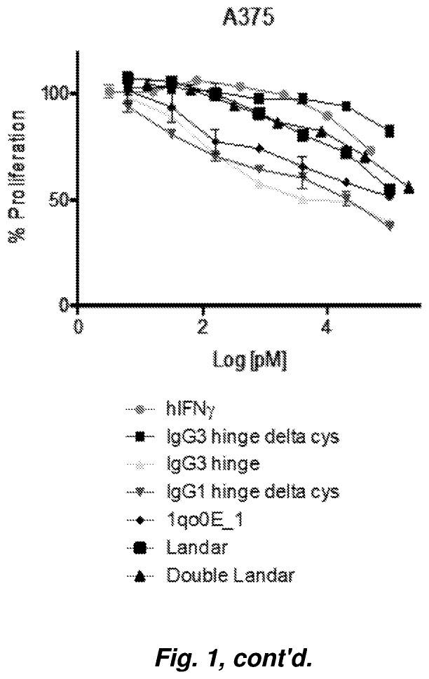

IFNγ was joined with the different linkers after the C_{H}3 domain of anti-CSPG4, transiently expressed in 293T cells and protein isolated from culture supernatants. The fusion proteins were then evaluated for their ability to inhibit the proliferation of OVCAR3, an ovarian cancer, T98MG, a glioma, and A375, a melanoma (FIG. 1). OVCAR3 and T98MG do not express CSPG-4, while A375 expresses it to high levels. Thus for OVCAR3 and T98MG we are examining the relative efficacy of untargeted fusion protein in comparison to untargeted recombinant IFNγ; for A375 we are comparing anti-CSPG-4 targeted IFNγ with untargeted recombinant IFNγ. For OVCAR3, the most effective IFNγ fusion utilized the IgG3 hinge with the second most effective using the IgG1 hinge Δ cys. Interestingly, both were more effective than this preparation of recombinant IFNγ.

In view of these, and other observations, in various embodiments, chimeric constructs are provided for the selective/specific delivery of active interferon gamma. In certain embodiments the chimeric construct(s) comprise a full-length immunoglobulin or a camelid antibody attached to an interferon gamma (IFNγ) where the immunoglobulin or camelid antibody is an antibody that binds to a tumor associated antigen (TAA), a first interferon gamma (IFNγ) is attached to a first constant heavy region 3 (CH3) of the immunoglobulin or camelid antibody by a first proteolysis resistant peptide linker, a second interferon gamma is attached to a second constant heavy region 3 (CH3) of the immunoglobulin or camelid antibody by a second proteolysis resistant peptide linker; and the first proteolysis resistant linker and the second proteolysis linker have a length and flexibility that permits the first interferon gamma and the second interferon gamma to dimerize.

In certain embodiments the first proteolysis resistant peptide linker and the second proteolysis peptide linker comprise or consist of amino acid sequences independently selected from the amino acid sequences of the peptide linkers shown in Table 1. Thus, for example, in one illustrative embodiment the linker comprises or consists of the amino acid sequence of the landar linker (SEQ ID NO:1). In another illustrative embodiment the linker comprises or consists of the amino acid sequence of the double landar linker (SEQ ID NO:2). In another illustrative embodiment the linker comprises or consists of the amino acid sequence of the 1qo0E_1 linker (SEQ ID NO:3). In another illustrative embodiment the linker comprises or consists of the amino acid sequence of the IgG3 hinge linker (SEQ ID NO:4). In still another illustrative embodiment the linker comprises or consists of the amino acid sequence of the IgG3 hinge delta cys (IgG3 hinge Δ cys) linker (SEQ ID NO:5). In yet still another illustrative embodiment the linker comprises or consists of the amino acid sequence of the IgG1 hinge delta cys (IgG1 hinge Δ cys) linker (SEQ ID NO:6).

As described in Example 1, when these cleavage resistant linkers are utilized to couple interferon gamma to antibody (or camelid antibody) CH3 domains the interferon gamma molecules are able to form an active dimer. The construct is thereby capable of specifically ro preferentially delivering the interferon gamma activity to a cell expressing a target that is bound by the antibody or camelid antibody.

The chimeric constructs and/or pharmaceutical formulations comprising the constructs are useful in the treatment of various cancers.

### Interferon Gamma.

In various embodiments the interferon gamma molecules used in the chimeric constructs described herein include full length human or murine interferon as well as truncated human or murine interferon and/or mutated human or murine interferon.

Interferon-gamma (IFNγ) is a cytokine produced by T-lymphocytes and natural killer cells and exists as a homodimer of two noncovalently bound polypeptide subunits. The sequence of the protein encoded by the human interferon gene is:

```
(SEQ ID NO: 13)
                10 20 30 40
MKYTSYILAF QLCIVLGSLG CYCQDPYVKE AENLKKYFNA
                50 60 70 80
GHSDVADNGT LFLGILKNWK EESDRKIMQS QIVSFYFKLF
                90 100 110 120
KNFKDDQSIQ KSVETIKEDM NVKFFNSNKK KRDDFEKLTN
              130 140 150 160
YSVTDLNVQR KAIHELIQVM AELSPAAKTG KRKRSQMLFR
GRRASQ
```

The first 23 amino acids are the leader sequence which is cleaved from the protein yielding a mature protein of 143 amino acids—but no methionine.

Each subunit has two potential N-glycosylation sites (Aggarwal et al. (1992) Human Cytokines, Blackwell Scientific Publications) at positions 25 and 97. Depending on the degree of glycosylation the molecular weight of IFNγ in dimer form is 34-50 kDa (Farrar et al. (1993) Ann. Rev. Immunol, 11: 571-611).

The primary sequence of wildtype human IFNγ (huIFNγ, aka huINFG) was reported by Gray et al. (1982) Nature 298: 859-863), Taya et al. (1982) EMBO J. 1: 953-958; Devos et al. (1982) Nucleic Acids Res. 10: 2487-2501; and Rinderknecht et al. (1984) J. Biol. Chem. 259: 6790-6797), and in EP 77670, EP 89676 and EP 110044. The 3D structure of huIFNγ was reported by Ealick et al. (1991) Science 252: 698-702, 1991).

Various naturally-occurring or mutated forms of the IFNγ subunit polypeptides have been reported, including one comprising a Cys-Tyr-Cys N-terminal amino acid sequence (positions (−3)-(−1) relative to SEQ ID NO:13), one comprising an N-terminal methionine (position −1 relative to SEQ ID NO:13), and various C-terminally truncated forms comprising 127-134 amino acid residues. It is known that 1-15 amino acid residues may be deleted from the C-terminus without abolishing IFNγ activity of the molecule. Furthermore, heterogeneity of the huIFNγ C-terminus was described by Pan et al. (1987) Eur. J. Biochem. 166: 145-149.

HuIFNγ muteins are reported by Slodowski et al. (1991) Eur. J. Biochem. 202:1133-1140, 1991, Luk et al. (1990) J. Biol. Chem. 265: 13314-13319, Seelig et al., (1988) Biochemistry 27: 1981-1987, Trousdale et al. (1985) Invest. Ophthalmol. Vis. Sci. 26: 1244-1251, and in EP 146354.

WO 1992/008737 discloses IFNγ variants comprising an added methionine in the N-terminal end of the full (residues 1-143) or partial (residues 1-132) amino acid sequence of wildtype human IFNG. EP 219 781 discloses partial huIFNγ sequences comprising amino 10 acid residues 3-124 (of SEQ ID NO:13)). U.S. Pat. No. 4,832,959 discloses partial huIFNγ sequences comprising residues 1-127, 5-146 and 5-127 of an amino acid sequence that compared to SEQ ID NO:13 has three additional N-terminal amino acid residues (CYC). U.S. Pat. No. 5,004,689 discloses a DNA sequence encoding huIFNγ without the 3 N-terminal amino acid residues CYC and its expression in E. coli. European patent EP 446582 discloses E. coli produced rhuIFNγ free of an 15 N-terminal methionine. U.S. Pat. No. 6,120,762 discloses a peptide fragment of huIFNγ comprising residues 95-134 thereof (relative to SEQ ID NO:13).

In various embodiments where interferon gamma is utilized in the constructs described herein the interferon gamma component(s) of the construct can be any polypeptide with IFNγ activity, and thus be derived from any origin, e.g. a non-human mammalian origin. However, in various embodiments, it is preferred that the parent polypeptide is huIFNγ, e.g., with the amino acid sequence shown in SEQ ID NO:13, or a variant or fragment thereof.

Examples of variants of hIFNγ that can be incorporated in the constructs contemplated herein are described above, and include, but are not limited to, e.g. huIFNγ with the N-terminal addition CYC, the cysteine modified variants described in U.S. Pat. No. 6,046,034, and the like. Thus, for example, in certain embodiments, the hIFNγ that can be incorporated in the constructs contemplated herein comprises cysteine substitutions at on or more of Glu8, Ser70, Ala18, His112, Lys81, Leu121, Gln49, and Leu96 (relative to the amino acid sequence of SEQ ID NO: 13). In certain embodiments the interferon gamma differs from the monomer of recombinant human interferon gamma in that at least one pair of amino acids from four predetermined amino acid pairs is exchanged for cysteine. These four amino acid pairs are Glu8 and Ser70, Ala18 and His 112, Lys81 and Leu121, and Gln-49 and Leu96 (see U.S. Pat. No. 6,046,034).

Additionally specific examples of fragments are those described above, and include, but are not limited to huIFNγ C-terminally truncated with 1-15 amino acid residues, e.g. with 1, 2, 3, 4, 5, 6, 7, 8, 9, 10, 11, 12, 13, 14, or 15 amino acid residues, and/or N-terminally truncated with 1-3 amino acid residues. In one illustrative, but non-limiting embodiment, the interferon comprises a truncated interferon consisting of the amino acid sequence:

(SEQ ID NO: 14)
DPYVKEAENL KKYFNAGHSD VADNGTLFLG
ILKNWKEESD RKIMQSQIVS FYFKLFKNFK
DDQSIQKSVE TIKEDMNVKF FNSNKKKRDD
FEKLTNYSVT DLNVQRKAIH ELIQVMAELS
PAAKTGKRKR SQM

In certain embodiments the use of chemically modified interferon is also contemplated. For example, in certain embodiments, the interferon is chemically modified to increase serum half-life. Thus, for example, (2-sulfo-9-fluorenylmethoxycarbonyl)₇-interferon-α2 undergoes time-dependent spontaneous hydrolysis, generating active interferon (see, e.g., Shechter et al. (2001) Proc. Natl. Acad. Sci., USA, 98(3): 1212-1217). Other modifications, include for example, N-terminal modifications in including, but not limited to the addition of PEG, protecting groups, and the like. U.S. Pat. No. 5,824,784, for example, describing N-terminally chemically modified interferon.

The foregoing interferons gamma(s) are intended to be illustrative and not limiting. Using the teaching provided herein, other suitable modified interferon gamma can readily be identified and produced.

### Antibodies (Targeting Moiety)

In various embodiments, the chimeric constructs described herein comprise a full-length antibody or a camelid antibody that specifically or preferentially binds a marker expressed by (e.g., on the surface of) or associated with the target cell(s). While essentially any cell can be targeted, certain preferred cells include those associated with a pathology characterized by hyperproliferation of a cell (i.e., a hyperproliferative disorder). Illustrative hyperproliferative disorders include, but are not limited to psoriasis, neutrophilia, polycythemia, thrombocytosis, and cancer.

Hyperproliferative disorders characterized as cancer include but are not limited to solid tumors, including, but not limited to cancers of the breast, respiratory tract, brain, reproductive organs, digestive tract, urinary tract, eye, liver, skin, head and neck, thyroid, parathyroid and their distant metastases. These disorders also include lymphomas, sarcomas, and leukemias. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to small-cell and non-small-cell lung carcinoma, as well as bronchial adenoma and pleuropulmonary blastoma. Examples of brain cancers include, but are not limited to brain stem and hypothalamic glioma, cerebellar and cerebral astrocytoma, medulloblastoma, ependymoma, as well as neuroectodermal and pineal tumor. Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small-intestine, and salivary gland cancers. Tumors of the urinary tract include, but are not limited to bladder, penile, kidney, renal pelvis, ureter, and urethral cancers. Eye cancers include, but are not limited to intraocular melanoma and retinoblastoma. Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to laryngeal/hypopharyngeal/nasopharyngeal/oropharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering the chimeric constructs described herein.

Accordingly, in certain embodiments, the antibody or camelid antibody specifically or preferentially binds a cancer marker (e.g., a tumor associated antigen). A wide variety of cancer markers are known to those of skill in the art. In certain embodiments, the markers need not be unique to cancer cells, but can also be effective where the expression of the marker is elevated in a cancer cell (as compared to normal healthy cells) or where the marker is not present at comparable levels in surrounding tissues (especially where the chimeric construct is delivered locally).

Illustrative cancer markers (tumor associated antigen(s) include, for example, chrondroitin sulfate proteoglycan 4 (CSPG4). Chrondroitin sulfate proteoglycan 4 (CSPG4) consisting of a protein core and a chondroitin sulfate side chain is also known as high-molecular weight melanoma associated antigen (HMW-MAA) and melanoma chondroitin sulface proteoglycan (MCSP). It has been studied as a target for the treatment of melanoma. This tumor antigen is highly expressed on greater than 80% of human melanomas and has a restricted distribution in normal tissues. CSPG4 plays an important role in the biology of melanoma cells through its modulation of integrin function and enhanced growth factor receptor-regulated pathways including sustained activation of ERK 1,2. It is also expressed on cancer-initiating cells and a broad range of other tumors including breast cancer including triple negative breast cancer, glioma, squamonous cell carcinoma of head and neck, myeloid leukemic cells, pancreatic carcinoma, chondrosarcoma, chordoma, mesothelioma, renal cell carcinoma, lung carcinoma, cancer stem cells, and ovarian carcinoma. Expression of CSPG4 is associated with the progression of many different cancers.

Another illustrative TAA is CD138 which is a marker associated with multiple myeloma (MM) cells, ovarian carcinoma, kidney carcinoma, gall bladder carcinoma, breast carcinoma, prostate cancer, lung cancer, colon carcinoma cells and cells of Hodgkin's and non-Hodgkin's lymphomas, chronic lymphocytic leukemia (CLL), acute lymphoblastic leukemia (ALL), acute myeloblastic leukemia (AML), solid tissue sarcomas, colon carcinomas as well as other hematologic malignancies and solid tumors that express CD138. Other cancers that have been shown to be positive for CD138 expression are many ovarian adenocarcinomas, transitional cell bladder carcinomas, kidney clear cell carcinomas, squamous cell lung carcinomas; breast carcinomas and uterine cancers.

Alpha-fetoprotein (AFP) is a marker for, inter alia, liver cancer and germ cell tumors, beta-2-microglobulin (B2M) is a marker for, inter alia, Multiple myeloma, chronic lymphocytic leukemia, and some lymphomas, beta-human chorionic gonadotropin (Beta-hCG) is a marker for, inter alia, choriocarcinoma and germ cell tumors, BRCA1 and BRCA2 gene mutations, are markers for, inter alia, ovarian cancer, c-kit/CD117 is a marker for, inter alia, gastrointestinal stromal tumor and mucosal melanoma, CA15-3/CA27.29 is a marker for, inter alia, breast cancer, CA19-9 is a marker for, inter alia, pancreatic cancer, gallbladder cancer, bile duct cancer, and gastric cancer, CA-125 is a marker for, inter alia, ovarian cancer, carcinoembryonic antigen (CEA) is a marker for, inter alia, colorectal cancer and some other cancers, CD20 is a marker for, inter alia, non-Hodgkin's lymphoma, chromogranin A (CgA) is a marker for, inter alia, neuroendocrine tumors, HE4 is a marker for, inter alia, ovarian cancer, HER2/neu is a marker for, inter alia, breast cancer, gastric cancer, and gastroesophageal junction adenocarcinoma, neuron-specific enolase (NSE) is a marker for, inter alia, small cell lung cancer and neuroblastoma, prostate-specific antigen (PSA) is a marker for, inter alia, prostate cancer, urokinase plasminogen activator (uPA) and plasminogen activator inhibitor (PAI-1) is a marker for, inter alia, breast cancer, and the like.

Additionally, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoprotein: CD46, CD55 and CD59, which have been found to be expressed on most tumor cells in vivo and in vitro. Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CDS, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-M1 marker. Various melanomas have been characterized by the HMB 45 marker. Non-hodgkins lymphomas have been characterized by the CD20, CD19, and Ia marker, and various prostate cancers have been characterized by the PSMA and SE10 markers.

In addition, many kinds of tumor cells display unusual antigens that are either inappropriate for the cell type and/or its environment, or are only normally present during the organisms' development (e.g. fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2). HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 2.

TABLE 2
Illustrative cancer markers and associated references, all of which are incorporated
herein by reference for the purpose of identifying the referenced tumor markers.

| Marker | Reference |
|---|---|
| CD138 | O'Connell et al. (2004) Am. J. Clin. Pathol., 121(2): 254-263 |
| CSPG4 | |
| α-fetoprotein | Esteban et al. (1996) Tumour Biol., 17(5): 299-305 |
| 5 alpha reductase | Délos et al. (1998) Int J Cancer, 75: 6 840-846 |
| 5T4 (or TPBG, | |
| trophoblast | |
| glycoprotein) | |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (\grave{ }998) J Exp Med., 188(6): 1185-1190 |
| B7 family members | Collins et al. (2005) Genome Biol., 6: 223.1-223.7 |
| BAGE | Böel et al. (1995) Immunity, 2: 167-175. |
| Bc12 | Koty et al. (1999) Lung Cancer, 23(2): 115-127 |
| bcr-abl (b3a2) | Verfaillie et al. (\grave{ }996) Blood, 87(11): 4770-4779 |
| CA-125 | Bast et al. (\grave{ }998) Int J Biol Markers, 13(4): 179-187 |
| CASP-8/FLICE | Mandruzzato et al. (1997) J Exp Med., 186(5): 785-793. |
| Cathepsins | Thomssen et al.(1995) Clin Cancer Res., 1(7): 741-746 |
| CD1 | |
| CD115 | |
| CD123 | |
| CD13 | |
| CD14 | |
| CD15 | |
| CD19 | Scheuermann et al. (1995) Leuk Lymphoma, 18(5-6): 385-397 |
| CD2 | |
| CD20 | Knox et al. (1996) Clin Cancer Res., 2(3): 457-470 |
| CD200 | |
| CD203c | |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD23 | |
| CD25 | |
| CD276 | Hofmeyer et al. (2008) Proc. Natl. Acad. Sci. USA, 105(30): |
| | 10277-10278 |
| CD3 | |
| CD30 | |
| CD303 | |
| CD33 | Nakase et al. (1996) Am J Clin Pathol., 105(6): 761-768 |
| CD34 | |
| CD35 | Yamakawa et al. Cancer, 73(11): 2808-2817 |
| CD37 | |
| CD38 | |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD56 | |
| CD59 (791Tgp72) | Jarvis et al. (1997) Int J Cancer, 71(6): 1049-1055 |
| CD7 | |
| CD70 | |
| CD74 | |
| CD79 | |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| CLL-1 | |
| c-MET (or HGFR) | |
| c-myc | Watson et al. (1991) Cancer Res., 51(15): 3996-4000 |
| Cox-2 | Tsujii et al. (1998) Cell, 93: 705-716 |
| Cripto | |
| DCC | Gotley et al. (1996) Oncogene, 13(4): 787-795 |
| DcR3 | Pitti et al. (1998) Nature, 396: 699-703 |
| DLL3 | |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| endoplasmin | U.S. Patent Application No. US20120009194 (Ferrone et al.) |
| EPCAM | |
| EphA2 | |
| EphB3 | |
| ETBR | |
| FcRL5 | |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| FOLR1 | |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| Glutathione | Hengstler (1998) et al. Recent Results Cancer Res., 154: 47-85 |
| S-transferase | |
| glycosphingolipid | |
| GD2 | |
| GnRH | Bahk et al.(1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239-247 |
| gp-100-in4 | Kirkin et al. (1998) APMIS, 106(7): 665-679 |
| gp15 | Maeurer et al.(1996) Melanoma Res., 6(1): 11-24 |
| gp75/TRP-1 | Lewis et al.(1995) Semin Cancer Biol., 6(6): 321-327 |
| GPNMB | |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| Her4 | |
| HLA-DR | |
| HM 1.24 | |
| HMB 45 | |
| HMTV | Kahl et al.(1991) Br J Cancer, 63(4): 534-540 |
| HMW-MAA | |
| Hsp70 | Jaattela et al. (1998) EMBO J., 17(21): 6124-6134 |
| hTERT (telomerase) | Vonderheide et al. (1999) Immunity, 10: 673-679. 1999. |
| IFN-α | Moradi et al. (1993) Cancer, 72(8): 2433-2440 |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| integrin | |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, | Abrams et al. (1996) Semin Oncol., 23(1): 118-134 |
| N-ras | |
| KSA (CO17-1A) | Zhang et al. (1998) Clin Cancer Res., 4(2): 295-302 |
| LDLR-FUT | Caruso et al. (1998) Oncol Rep., 5(4): 927-930 |
| Leu-M1 | |
| Lewis A like | |
| carbohydrate | |
| Lewis Y | |
| LIV1 | |
| MAGE Family | Marchand et al. (1999) Int J Cancer, 80(2): 219-230 |
| (MAGE1, MAGE3, | |
| etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/MART-1 | Lewis and Houghton (1995) Semin Cancer Biol., 6(6): 321-327 |
| mesothelin | Chang et al. (1996) Proc. Natl. Acad. Sci., USA, 93(1): 136-140 |
| MIC A/B | Groh et al. (1998) Science, 279: 1737-1740 |
| MN | |
| Mox1 | Candia et al. (1992) Development, 116(4): 1123-1136 |
| MT-MMP's, such as | Sato and Seiki (1996) J Biochem (Tokyo), 119(2): 209-215 |
| MMP2, MMP3, | |
| MMP7, MMP9 | |
| MUC16 | |
| Mucin, such as MUC-1, | Lewis and Houghton (1995) Semin Cancer Biol., 6(6): 321-327 |
| MUC-2, MUC-3, | |
| MUC-4, MUC-16 | |
| MUM-1 | Kirkin et al. (1998) APMIS, 106(7): 665-679 |
| NaPi2b | |
| Nectin-4 | |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| p16INK4 | Quelle et al. (1995) Oncogene Aug. 17, 1995; 11(4): 635-645 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PMSA | |
| PRAME | Kirkin et al. (1998) APMIS, 106(7): 665-679 |
| Probasin | Matuo et al. (1985) Biochem Biophys Res Commun., 130(1): 293-300 |
| Progenipoietin | — |
| PSA (phosphatidyl | Sanda et al. (1999) Urology, 53(2): 260-266. |
| serine antigen) | |
| PSM | Kawakami et al.(1997) Cancer Res., 57(12): 2321-2324 |
| RAGE-1 | Gaugler et al.(1996) Immunogenetics, 44(5): 323-330 |
| Rb | Dosaka-Akita et al. (1997) Cancer, 79(7): 1329-1337 |
| RCAS1 | Sonoda et al.(1996) Cancer, 77(8): 1501-1509. |
| SART-1 | Kikuchi et al.(1999(Int J Cancer, 81(3): 459-466 |
| SE10 | |
| SIRP.alpha. | |
| SLAM family | |
| members | |
| SLC44A4 | |
| SSX gene | Gure et al. (1997) Int J Cancer, 72(6): 965-971 |
| family | |
| STAT3 | Bromberg et al. (1999) Cell, 98(3): 295-303 |
| STEAP-1 | |
| STn (mucin assoc.) | Sandmaier et al. (1999) J Immunother., 22(1): 54-66 |
| TAG-72 | Kuroki et al. (1990)Cancer Res., 50(16): 4872-4879 |
| TF (or tissue factor) | |
| TGF-α | Imanishi et al. (1989) Br J Cancer, 59(5): 761-765 |
| TGF-β | Picon et al. (1998) Cancer Epidemiol Biomarkers Prey, 7(6): |
| | 497-504 |
| Thymosin β 15 | Bao et al. (1996) Nature Medicine. 2(12), 1322-1328 |
| TNF superfamily | Iqbal Grewal, ed. Therapeutic Targets of the TNF Superfamily”, |
| members | Landes Bioscience/Springer Science + Business Media, LLC dual |
| | imprint/Springer series: Advances in Experimental Medicine and |
| | Biology, 2009) |
| TPA | Maulard et al. (1994) Cancer, 73(2): 394-398 |
| TPI | Nishida et al.(1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| VLA | |
| ZAG | Sanchez et al. (1999) Science, 283(5409): 1914-1919 |
| β-catenin | Hugh et al. (1999) Int J Cancer, 82(4): 504-11 |

Antibodies directed against (that preferentially or specifically bind) any of the foregoing markers can be used in the chimeric interferon gamma constructs described herein. In certain embodiments the target markers include, but are not limited to CD138, CSPG4, members of the epidermal growth factor family (e.g., HER2, HER3, EGF, HER4), CD1, CD2, CD3, CDS, CD7, CD13, CD14, CD15, CD19, CD20, CD21, CD23, CD25, CD33, CD34, CD38, 5E10, CEA, HLA-DR, HM 1.24, HMB 45, 1a, Leu-M1, MUC1, PMSA, TAG-72, phosphatidyl serine antigen, and the like.

Antibodies that specifically or preferentially bind tumor markers are well known to those of skill in the art and may are commercially available or the amino acid sequences thereof are well known and can readily be used to fabricate the antibody using methods well known to those of skill in the art.

Antibodies that bind to CSPG4 include, but are not limited to VF1-TP34, VF1-TP34, VF1-TP41.2, TP61.5, 9.2.27, 149.53, 149.53, 225.28, 225.28s, 763.74, and scFv-FcC21 (see, e.g., PCT Pub No: WO 2014/194100 (PCT/US2014/040036).

Antibodies that specifically or preferentially bind CD138 are well known to those of skill in the art and many are commercially available. For example Wijdenes et al. (1996) British J. Haematol. 94, 318-323 describe an antibody that is specific for CD138 (syndecan-1) and this antibody is commercially available from Abcam, Miltenyi Biotec, and the like. Other illustrative and non-limiting anti-CD138 antibodies include, but are not limited to the polyclonal rabbit anti-human CD138 antibody LS-B3341 and the monoclonal mouse anti-Human CD138 Antibody LS-B4051 available from LifeSpan Biosciences, Inc., monoclonal antibody (MI15) available from Pierce Antibodies, Biotest BT-062 anti-CD138, and the like. Other anti-CD138 antibodies include, but are not limited to B-B2, 1D4, nBT062, B-B4, BC/B-B4, B-B2, DL-101, 1 D4, MI15, 1.BB.210, 2Q1484, 5F7, 104-9, and 281-2 (see, e.g., Gattei et al. (1999) British J. Haematol., 104(1): 152-162, and U.S. Patent Pub No: 2009/0175863).

Antibodies to CD33 include for example, HuM195 (see, e.g., Kossman et al. (1999) Clin. Cancer Res. 5: 2748-2755), CMA-676 (see, e.g., Sievers et al., (1999) Blood 93: 3678-3684).

Antibodies to CD38 include for example, AT13/5 (see, e.g., Ellis et al. (1995) J. Immunol. 155: 925-937), HB7, and the like.

Antibodies been developed against Her-2/neu, include, but are not limited to trastuzumab (e.g., HERCEPTIN®; Fornier et al. (1999) Oncology (Huntingt) 13: 647-58), TAB-250 (Rosenblum et al. (1999) Clin. Cancer Res. 5: 865-874), BACH-250 (Id.), TA1 (Maier et al. (1991) Cancer Res. 51: 5361-5369), and the mAbs described in U.S. Pat. Nos. 5,772,997; 5,770,195 (mAb 4D5; ATCC CRL 10463); and U.S. Pat. No. 5,677,171.

Other fully human anti-HER2/neu antibodies are well known to those of skill in the art. Such antibodies include, but are not limited to the C6 antibodies such as C6.5, DPLS, G98A, C6MH3-B1, B1D2, C6VLB, C6VLD, C6VLE, C6VLF, C6MH3-D7, C6MH3-D6, C6MH3-D5, C6MH3-D3, C6MH3-D2, C6MH3-D1, C6MH3-C4, C6MH3-C3, C6MH3-B9, C6MH3-B5, C6MH3-B48, C6MH3-B47, C6MH3-B46, C6MH3-B43, C6MH3-B41, C6MH3-B39, C6MH3-B34, C6MH3-B33, C6MH3-B31, C6MH3-B27, C6MH3-B25, C6MH3-B21, C6MH3-B20, C6MH3-B2, C6MH3-B16, C6MH3-B15, C6MH3-B11, C6MH3-B1, C6MH3-A3, C6MH3-A2, and C6ML3-9. These and other anti-HER2/neu antibodies are described in U.S. Pat. Nos. 6,512,097 and 5,977,322, in PCT Publication WO 97/00271, in Schier et al. (1996) J Mol Biol 255: 28-43, Schier et al. (1996) J Mol Biol 263: 551-567, and the like.

Illustrative anti-MUC-1 antibodies include, but are not limited to Mc5 (see, e.g., Peterson et al. (1997) Cancer Res. 57: 1103-1108; Ozzello et al. (1993) Breast Cancer Res. Treat. 25: 265-276), and hCTMO1 (see, e.g., Van Hof et al. (1996) Cancer Res. 56: 5179-5185).

Illustrative anti-TAG-72 antibodies include, but are not limited to CC49 (see, e.g., Pavlinkova et al. (1999) Clin. Cancer Res. 5: 2613-2619), B72.3 (see, e.g., Divgi et al. (1994) Nucl. Med. Biol. 21: 9-15), and those disclosed in U.S. Pat. No. 5,976,531.

Illustrative anti-HM1.24 antibodies include, but are not limited to a mouse monoclonal anti-HM1.24 IgG_{2a/κ} and a humanized anti-HM1.24 IgG₁/κ antibody (see, e.g., Ono et al. (1999) Mol. Immunol. 36: 387-395).

Antibodies directed to various members of the epidermal growth factor receptor family include, but are not limited to anti-EGF-R antibodies as described in U.S. Pat. Nos. 5,844,093 and 5,558,864, and in European Patent No. 706,799A). Other illustrative anti-EGFR family antibodies include, but are not limited to antibodies such as C6.5, C6ML3-9, C6MH3-B1, C6-B1D2, F5, HER3.A5, HER3.F4, HER3.H1, HER3.H3, HER3.E12, HER3.B12, EGFR.E12, EGFR.C10, EGFR.B11, EGFR.E8, HER4.B4, HER4.G4, HER4.F4, HER4.A8, HER4.B6, HER4.D4, HER4.D7, HER4.D11, HER4.D12, HER4.E3, HER4.E7, HER4.F8 and HER4.C7 and the like (see, e.g., U.S. Patent publications US 2006/0099205 A1 and US 2004/0071696 A1 which are incorporated herein by reference).

As described in U.S. Pat. Nos. 6,512,097 and 5,977,322 other anti-EGFR family member antibodies can readily be produced by shuffling light and/or heavy chains followed by one or more rounds of affinity selection. Thus in certain embodiments, this invention contemplates the use of one, two, or three CDRs in the VL and/or VH region that are CDRs described in the above-identified antibodies and/or the above identified publications.

Anti-CD20 antibodies are well known to those of skill and include, but are not limited to rituximab, Ibritumomab tiuxetan, and tositumomab, AME-133v (Applied Molecular Evolution), Ocrelizumab (Roche), Ofatumumab (Genmab), TRU-015 (Trubion) and IN/MU-106 (Immunomedics).

In certain embodiments the antibody is an anti-CD138 which comprises a heavy chain having an amino acid sequence as set forth in SEQ ID NO:15:

(SEQ ID NO: 15)
MGWSYIILFLVATATGVHSQVQLQQSGSELMMPGA
SVKISCKATGYTFSNYWIEWVKQRPGHGLEWIGEI
LPGTGRTIYNEKFKGKATFTADISSNTVQMQLSSL
TSEDSAVYYCARRDYYGNFYYAMDYWGQGTSVTVS
S

and the light chain having an amino acid sequence as set forth in SEQ ID NO:16:

(SEQ ID NO: 16)
MKSQTQVFIFLLLCVSGAHGDIQMTQSTSSLSASL
GDRVTISCSASQGINNYLNWYQQKPDGTVELLIYY
TSTLQSGVPSRFSGSGSGTDYSLTISNLEPEDIGT
YYCQQYSKLPRTFGGGTKLEIK.

Construction of such an antibody is described in U.S. Pat. No. 9,803,021, and in PCT Publication No: WO 2014/089354 (PCT/US2013/073410), which are incorporated herein by reference for the antibodies described therein.

In certain embodiments the antibody is an anti-Her2/neu antibody which comprises the heavy chain having an amino acid sequence as set forth in SEQ ID NO:17:

(SEQ ID NO: 17)
MECSWVMLFLLSVTAGVHSEVQLVESGGGLVQPGG
SLRLSCAASGFNIKDTYIHWVRQAPGKGLEWVARI
YPTNGYTRYADSVKGRFTISADTSKNTAYLQMNSL
RAEDTAVYYCSRWGGDGFYAMDYWGQGTLVTVSSA
STKGPSVFPLAPSSKSTSGGTAALGCLVKDYFPEP
VTVSWNSGALTSGVHTFPAVLQSSGLYSLSSVVTV
PSSSLGTQTYICNVNHKPSNTKVDKKVEPKSCDKT
HTCPPCPAPELLGGPSVFLFPPKPKDTLMISRTPE
VTCVVVDVSHEDPEVKFNWYVDGVEVHNAKTKPRE
EQYNSTYRVVSVLTVLHQDWLNGKEYKCKVSNKAL
PAPIEKTISKAKGQPREPQVYTLPPSRDELTKNQV
SLTCLVKGFYPSDIAVEWESNGQPENNYKTTPPVL
DSDGSFFLYSKLTVDKSRWQQGNVFSCSVMHEALH
NHYTQKSLSLSPGK

where amino acid residues 1-19 represent a signal peptide; and the light chain having an amino acid sequence as set forth in SEQ ID NO:18:

(SEQ ID NO: 18)
MEWSCVMLFLLSVTAGVHSDIQMTQSPSSLSASVG
DRVTITCRASQDVNTAVAWYQQKPGKAPKLLIYSA
SFLYSGVPSRFSGSRSGTDFTLTISSLQPEDFATY
YCQQHYTTPPTFGQGTKVEIKRTVAAPSVFIFEPS
DEQLKSGTASVVCLLNNFYPREAKVQWKVDNALQS
GNSQESVTEQDSKDSTYSLSSTLTLSKADYEKHKV
YACEVTHQGLSSPVTKSFNRGEC

where amino acid residues 1-19 represent a signal peptide.

In certain embodiments, the antibody is an anti-CD20 antibody which comprises the heavy chain having an amino acid sequence as set forth in SEQ ID NO:19:

(SEQ ID NO: 19)
MYLGLNCVIIVFLLKGVQSQVQLQQPGAELVKPGA
SVKMSCKASGYTFTSYNMHWVKQTPGRGLEWIGAI
YPGNGDTSYNQKFKGKATLTADKSSSTAYMQLSSL
TSEDSAVYYCARSTYYGGDWYFNVWGAGTTVTVSA
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCDK
THTCPPCPAPELLGGPSVFLFPPKPKDTLMISRTP
EVTCVVVDVSHEDPEVKFNWYVDGVEVHNAKTKPR
EEQYNSTYRVVSVLTVLHQDWLNGKEYKCKVSNKA
LPAPIEKTISKAKGQPREPQVYTLPPSRDELTKNQ
VSLTCLVKGFYPSDIAVEWESNGQPENNYKTTPPV
LDSDGSFFLYSKLTVDKSRWQQGNVFSCSVMHEAL
HNHYTQKSLSLSPGK

where amino acid residues 1-19 represent a signal peptide; and the light chain having an amino acid sequence as set forth in SEQ ID NO:20:

(SEQ ID NO: 20)
MKLPVRLLVLMFWIPASSSQIVLSQSPAILSASPG
EKVTMTCRASSSVSYIHWFQQKPGSSPKPWIYATS
NLASGVPVRFSGSGSGTSYSLTISRVEAEDAATYY
CQQWTSNPPTFGGGTKLEIKRTVAAPSVFIFPPSD
EQLKSGTASVVCLLNNFYPREAKVQWKVDNALQSG
NSQESVTEQDSKDSTYSLSSTLTLSKADYEKHKVY
ACEVTHQGLSSPVTKSFNRGEC

wherein amino acid residues 1-19 represent a signal peptide.

In certain embodiments the antibody is an anti-endoplasmin antibody which comprises the heavy chain having an amino acid sequence as set forth in SEQ ID NO:21:

(SEQ ID NO: 21)
MYLGLNCVIIVFLLKGVQSQVQLVQSGAEVKKPGA
SVKVSCKASGYTFTSYAMHWVRQAPGQRLEWMGWI
NAGNGNTKYSQKFQGRVTITRDTSASTAYMELSSL
RSEDTAVYYCARAHFDYWGQGTLVTVSAASTKGPS
VFPLAPSSKSTSGGTAALGCLVKDYFPEPVTVSWN
SGALTSGVHTFPAVLQSSGLYSLSSVVTVPSSSLG
TQTYICNVNHKPSNTKVDKKVEPKSCDKTHTCPPC
PAPELLGGPSVFLFPPKPKDTLMISRTPEVTCVVV
DVSHEDPEVKFNWYVDGVEVHNAKTKPREEQYNST
YRVVSVLTVLHQDWLNGKEYKCKVSNKALPAPIEK
TISKAKGQPREPQVYTLPPSRDELTKNQVSLTCLV
KGFYPSDIAVEWESNGQPENNYKTTPPVLDSDGSF
FLYSKLTVDKSRWQQGNVFSCSVMHEALHNHYTQK
SLSLSPGK

where amino acid residues 1-19 represent a signal peptide; and the light chain having an amino acid sequence as set forth in SEQ ID NO:22:

(SEQ ID NO: 22)
MEAPAQLLFLLLLWLPDTTGEIELTQSPSSLSASV
GDRVTITCRASQSISSYLNWYQQKPGKAPKLLIYA
ASSLQSGVPSRFSGSGSGTDFTLTISSLQPEDFAT
YYCQQSYSTPPTFGQGTKVEIKRTVAAPSVFIFPP
SDEQLKSGTASVVCLLNNFYPREAKVQWKVDNALQ
SGNSQESVTEQDSKDSTYSLSSTLTLSKADYEKHK
VYACEVTHQGLSSPVTKSFNRGEC

where amino acid residues 1-20 represent a signal peptide.

In certain embodiments the antibody is an anti-CD33 antibody which comprises the heavy chain having an amino acid sequence as set forth in SEQ ID NO:23:

(SEQ ID NO: 23)
MEWSWVFLFFLSVTTGVHSQVQLVQSGAEVKKPGS
SVKVSCKASGYTITDSNIHWVRQAPGQSLEWIGYI
YPYNGGTDYNQKFKNRATLTVDNPTNTAYMELSSL
RSEDTAFYYCVNGNPWLAYWGQGTLVTVSSASTKG
PSVFPLAPSSKSTSGGTAALGCLVKDYFPEPVTVS
WNSGALTSGVHTFPAVLQSSGLYSLSSVVTVPSSS
LGTQTYICNVNHKPSNTKVDKKVEPKSCDKTHTCP
PCPAPELLGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALPAPI
EKTISKAKGQPREPQVYTLPPSRDELTKNQVSLTC
LVKGFYPSDIAVEWESNGQPENNYKTTPPVLDSDG
SFFLYSKLTVDKSRWQQGNVFSCSVMHEALHNHYT
QKSLSLSPGK

where amino acid residues 1-19 represent a signal peptide; and the light chain having an amino acid sequence as set forth in SEQ ID NO: 24:

(SEQ ID NO: 24)
MSVPTQVLGLLLLWLTDARCDIQLTQSPSTLSASV
GDRVTITCRASESLDNYGIRFLTWFQQKPGKAPKL
LMYAASNQGSGVPSRFSGSGSGTEFTLTISSLQPD
DFATYYCQQTKEVPWSFGQGTKVEVKRTVAAPSVF
IFPPSDEQLKSGTASVVCLLNNFYPREAKVQWKVD
NALQSGNSQESVTEQDSKDSTYSLSSTLTLSKADY
EKHKVYACEVTHQGLSSPVTKSFNRGEC

where amino acid residues 1-20 represent a signal peptide.

In certain embodiments the antibody is an anti-CD276 antibody that comprises a heavy chain variable region having an amino acid sequence as set forth in SEQ ID NO:25:

(SEQ ID NO: 25)
MNFGFRLIFLALILKGVQCEVQLVESGGGLVKPGG
SLKLSCEASRFTFSSYAMSWVRQTPEKRLEWVAAI
SGGGRYTYYPDSMKGRFTISRDNAKNFLYLQMSSL
RSEDTAMYYCARHYDGYLDYWGQGTTLTVSSAKTT
APSVYPLAPGSL

where amino acid residues 1-19 represent a signal peptide; and the light chain having an amino acid sequence as set forth in SEQ ID NO: 26:

(SEQ ID NO: 26)
MKSQSQVFVFVFLWLSGVDGDIVMTQFAGVDGDIV
MTQSHKFMSTSVGDRVSITCKASQDVSTTVAWYQQ
KPGQSPKLLIYSASYRYTGVPDRFTGSGSGTDFTF
TISSVQAEDLAVYYCQQHYSTPPTFGGGTKLEIKR
ADAAPTVSIFPPSSKLG

wherein amino acid residues 1-20 represent a signal peptide.

In various embodiments the full-length antibodies (immunoglobulins) used in the chimeric constructs described herein include, but are not limited to an IgA, IgD, IgE, IgG or IgM antibody. In certain embodiments the antibody can comprise kappa (κ) light chains or lambda (λ) light chains. In various embodiments, the IgG antibody can be an IgG1, IgG2, IgG3 or IgG4 antibody. In certain embodiments the antibody binds to a(n) antigen target that is expressed in or on the cell membrane (e.g., on the cell surface) of a tumor cell. In certain embodiments the antibody is an IgG antibody, e.g., an IgG1 antibody, more particularly, an IgG1 antibody having kappa light chains.

In certain embodiments the antibodies described herein (full-length immunoglobulins) can comprise a human antibody, a humanized antibody, or a chimeric antibody. In certain embodiments the antibody can be a non-human antibody (e.g., a murine antibody).

Humanized antibodies are antibodies that contain sequences derived from a human-antibody and from a non-human antibody. Suitable methods for humanizing antibodies include CDR-grafting (complementarity determining region grafting) (EP 0 239 400; WO 91/09967; U.S. Pat. Nos. 5,530,101; and 5,585,089), veneering or resurfacing (EP 0 592 106; EP 0 519 596; Padlan (1991) Mol. Immunol., 28: 489-498; Studnicka et al. (1994) Protein Eng., 7(6: 805-814; Roguska et al. (1994) Proc. Natl. Acad. Sci. USA, 91: 969-973; and the like), chain shuffling (U.S. Pat. No. 5,565,332) and DeImmunosation™ (Biovation, LTD). In CDR-grafting, the mouse (or other non-human species) complementarity-determining regions (CDRs) from, for example, mAb B-B4 are grafted into human variable frameworks, which are then joined to human constant regions, to create a human B-B4 antibody (hB-B4). Several antibodies humanized by CDR-grafting are now in clinical use, including MYLOTARG (Sievers et al. (2001) J. Clin. Oncol. 19: 3244-3254) and HECEPTIN (Pegram et al. (1998) J. Clin. Oncol., 16: 2659-2671).

The resurfacing technology uses a combination of molecular modeling, statistical analysis and mutagenesis to alter the non-CDR surfaces of antibody variable regions to resemble the surfaces of known antibodies of the target host. Strategies and methods for the resurfacing of antibodies, and other methods for reducing immunogenicity of antibodies within a different host, are disclosed, for example, in U.S. Pat. No. 5,639,641. Human antibodies can be made by a variety of methods known in the art including phage display methods. See also U.S. Pat. Nos. 4,444,887, 4,716,111, 5,545,806, and 5,814,318; and international patent application publications WO 98/46645, WO 98/50433, WO 98/24893, WO 98/16654, WO 96/34096, WO 96/33735, and WO 91/10741.

Antibodies that have undergone any non-natural modification such as chimeric human/mouse antibodies or a chimeric human/monkey antibodies, humanized antibodies or antibodies that were engineered to, for example, improve their affinity to the target cells or diminish their immunogenicity are also contemplated.

Chimerized antibodies, maintain the antibody binding region of the non-human antibody, e.g., the murine antibody they are based on, while any constant regions may be provided for by, e.g., a human antibody. Generally, chimerization and/or the exchange of constant regions of an antibody will not affect the affinity of an antibody because the regions of the antibody that contribute to antigen binding are not affected by this exchange. In a preferred embodiment, the engineered, in particular chimerized, antibody may have a higher binding affinity (as expressed by K_{D} values) than the respective non-human antibody it is based on. For example, the anti-CD138 antibody nBT062 antibody and antibodies based thereon may have higher antibody affinity than the murine B-B4 on which they are based.

In various embodiments fully human antibodies may also be used. Those antibodies can be selected by the phage display approach, where desired TAA (e.g., CD138, CSPG4, etc.) is used to selectively bind phage expressing, for example, B-B4 variable regions. This approach can be advantageously coupled with an affinity maturation technique to improve the affinity of the antibody.

In various embodiments camelid antibodies are also contemplated. Camelid and shark antibodies comprise a homodimeric pair of two chains of V-like and C-like domains (neither has a light chain). Since the VH region of a heavy chain dimer IgG in a camelid does not have to make hydrophobic interactions with a light chain, the region in the heavy chain that normally contacts a light chain is changed to hydrophilic amino acid residues in a camelid. VH domains of heavy-chain dimer IgGs are called VHH domains. Shark Ig-NARs comprise a homodimer of one variable domain (termed a V-NAR domain) and five C-like constant domains (C-NAR domains).

In camelids, the diversity of antibody repertoire is determined by the complementary determining regions (CDR) 1, 2, and 3 in the VH or VHH regions. The CDR3 in the camel VHH region is characterized by its relatively long length averaging 16 amino acids (see, e.g., Muyldermans et al. (1994) Prog. Engin. 7(9): 1129). This is in contrast to CDR3 regions of antibodies of many other species. For example, the CDR3 of mouse VH has an average of 9 amino acids. Libraries of camelid-derived antibody variable regions, that maintain the in vivo diversity of the variable regions of a camelid, can be made by, for example, the methods disclosed in U.S. Patent Pub. No. 2005/0037421

In certain embodiments, the antibody or camelid antibody has an affinity (K_{D}) for the tumor associated antigen (TAA) (e.g., CSPG4, CD138, Her2/neu, etc.) of at least 1×10⁻⁶ M, or at least 1×10⁻⁷ M, or at least 1×10⁻⁸ M, or at least 1×10⁻⁹ M, or at least 1×10⁻¹⁰ M, or at least 1×10⁻¹¹ M.

The foregoing antibodies for use in the chimeric constructs described herein are illustrative and non-limiting. Using the teachings provided herein, numerous other antibodies will be available to one of skill in the art.

### Treatment of Cancers Using Chimeric Constructs.

In certain embodiments the chimeric constructs (targeted interferon gamma constructs) described herein can be used for the treatment or prophylaxis of cancer.

Illustrative cancers include any cancer or cancer cell that is responsive to treatment with interferon gamma. Additionally, interferon gamma helps turn on the immune response and can be useful even if the cancer is not directly responsive because the host anti-tumor response if turned on (e.g., upregulated). Illustrative cancers include, but are not limited to g of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma (olofactory neuroblastoma), extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

### Pharmaceutical Formulations.

The chimeric constructs described herein are useful for parenteral, topical, oral, or local administration (e.g. injected into a tumor site), aerosol administration, or transdermal administration, for prophylactic, but principally for therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the antibodies described herein and/or immunoconjugates thereof and pharmaceutical compositions comprising antibodies described herein and/or immunoconjugates thereof, when administered orally, are preferably protected from digestion. This can be accomplished by a number of means known to those of skill in the art, e.g., by complexing the protein with a composition to render it resistant to acidic and enzymatic hydrolysis or by packaging the protein in an appropriately resistant carrier such as a liposome. Means of protecting proteins from digestion are well known in the art.

In various embodiments a composition, e.g., a pharmaceutical composition, containing one or more chimeric constructs described herein, formulated together with a pharmaceutically acceptable carrier are provided.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, may be coated in a material to protect the compound from the action of acids and other natural conditions that may inactivate the compound.

In certain embodiments one or more chimeric constructs described herein can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

By way of illustration, a pharmaceutically acceptable salt can be prepared for chimeric constructs described herein having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines Salts may also be formed with caffeine, tromethamine and similar molecules.

Methods of formulating pharmaceutically active agents as salts, esters, amide, prodrugs, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of one or more chimeric constructs described herein are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the chimeric constructs described herein. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

Pharmaceutical compositions comprising one or more chimeric constructs described herein can be administered alone or in combination therapy, i.e., combined with other agents. For example, the combination therapy can include one or more chimeric constructs described herein and at least one or more additional therapeutic agents. The pharmaceutical compositions can also be administered in conjunction with radiation therapy and/or surgery.

A composition comprising one or more chimeric constructs described herein can be administered by a variety of methods known in the art. As will be appreciated by the skilled artisan, the route and/or mode of administration will vary depending upon the desired results. The chimeric constructs described herein can be prepared with carriers that will protect the chimeric constructs against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see, e.g., Sustained and Controlled Release Drug Delivery Systems, J. R. Robinson, ed., Marcel Dekker, Inc., New York, 1978).

In certain embodiments administration of one or more chimeric constructs described herein may be facilitated by coating the construct(s) or co-administering the constructs with a material to prevent its inactivation. For example, the construct(s) may be administered to a subject in an appropriate carrier, for example, liposomes, or a diluent. Pharmaceutically acceptable diluents include, but are not limited to, saline and aqueous buffer solutions. Liposomes include, but are not limited to, water-in-oil-in-water CGF emulsions as well as conventional liposomes (Strejan et al. (1984) J. Neuroimmunol, 7: 27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of is contemplated. Supplementary active compounds can also be incorporated into the compositions.

In various embodiments the one or more chimeric constructs described herein are typically sterile and stable under the conditions of manufacture and storage. The composition(s) can be formulated as a solution, a microemulsion, in a lipid or liposome, or other ordered structure suitable to contain high drug concentration(s). In certain embodiments the carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the construct(s) described herein in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, illustrative methods of preparation include vacuum drying, and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, in certain embodiments, the one or more chimeric constructs described herein may be administered once or twice daily, or once or twice weekly, or once or twice monthly by subcutaneous injection.

It is especially advantageous to formulate parenteral compositions in unit dosage form for ease of administration and uniformity of dosage. Unit dosage form as used herein refers to physically discrete units suited as unitary dosages for the subjects to be treated. Each unit contains a predetermined quantity of active compound calculated to produce the desired therapeutic effect in association with the required pharmaceutical carrier. The specifications for the unit dosage forms are dictated by and directly dependent on (a) the unique characteristics of the construct(s) and the particular therapeutic effect to be achieved, and (b) the limitations inherent in the art of compounding such construct(s) for the treatment of individuals.

In certain embodiments the formulation comprises a pharmaceutically acceptable anti-oxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of one or more chimeric constructs described herein include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient (chemeric construct(s)) that can be combined with a carrier material to produce a single dosage form will vary depending upon the subject being treated, and the particular mode of administration. The amount of active ingredient that can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

Formulations of one or more chimeric constructs described herein that are suitable for vaginal administration also include pessaries, tampons, creams, gels, pastes, foams or spray formulations containing such carriers as are known in the art to be appropriate. Dosage forms for the topical or transdermal administration of antibodies and/or immunoconjugates described herein include powders, sprays, ointments, pastes, creams, lotions, gels, solutions, patches and inhalants. In certain embodiments the active compound may be mixed under sterile conditions with a pharmaceutically acceptable carrier, and with any preservatives, buffers, or propellants that may be required.

The phrases “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

Examples of suitable aqueous and nonaqueous carriers that may be employed in the pharmaceutical compositions comprising one or more chimeric constructs described herein include, but are not limited to water, ethanol, polyols (such as glycerol, propylene glycol, polyethylene glycol, and the like), and suitable mixtures thereof, vegetable oils, such as olive oil, and injectable organic esters, such as ethyl oleate, and the like. Proper fluidity can be maintained, for example, by the use of coating materials, such as lecithin, by the maintenance of the required particle size in the case of dispersions, and by the use of surfactants.

In various embodiments these compositions may also contain adjuvants such as preservatives, wetting agents, emulsifying agents and dispersing agents. Particular examples of adjuvants that are well-known in the art include, for example, inorganic adjuvants (such as aluminum salts, e.g., aluminum phosphate and aluminum hydroxide), organic adjuvants (e.g., squalene), oil-based adjuvants, virosomes (e.g., virosomes that contain a membrane-bound hemagglutinin and neuraminidase derived from the influenza virus).

Prevention of presence of microorganisms in formulations may be ensured both by sterilization procedures, and/or by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

When the antibodies and/or immunoconjugates described herein are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the antibodies and/or immunoconjugates described herein, that may be used in a suitable hydrated form, and/or the pharmaceutical compositions, are formulated into pharmaceutically acceptable dosage forms by conventional methods known to those of skill in the art.

Actual dosage levels of the active ingredients (e.g., antibodies and/or immunoconjugates described herein) in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the constructs of the invention employed in the pharmaceutical composition at levels lower than that required in order to achieve the desired therapeutic effect and gradually increase the dosage until the desired effect is achieved. In general, a suitable daily dose of antibodies and/or immunoconjugates described herein will be that amount of the construct that is the lowest dose effective to produce a therapeutic effect. Such an effective dose will generally depend upon the factors described above. In certain embodiments, it is preferred that administration be intravenous, intramuscular, intraperitoneal, or subcutaneous, preferably administered proximal to the site of the target. If desired, the effective daily dose of a therapeutic composition may be administered a single dosage, or as two, three, four, five, six or more sub-doses administered separately at appropriate intervals throughout the day, optionally, in unit dosage forms. While it is possible for antibodies and/or immunoconjugates described herein to be administered alone, it is typically preferable to administer the compound(s) as a pharmaceutical formulation (composition).

In certain embodiments the antibodies and/or immunoconjugates described herein can be administered with medical devices known in the art. For example, in a illustrative embodiment, antibodies and/or immunoconjugates described herein can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of useful well-known implants and modules are described for example in U.S. Pat. No. 4,487,603, which discloses an implantable micro-infusion pump for dispensing medication at a controlled rate, in U.S. Pat. No. 4,486,194, which discloses a therapeutic device for administering medications through the skin, in U.S. Pat. No. 4,447,233, which discloses a medication infusion pump for delivering medication at a precise infusion rate, in U.S. Pat. No. 4,447,224, which discloses a variable flow implantable infusion apparatus for continuous drug delivery, in U.S. Pat. No. 4,439,196, which discloses an osmotic drug delivery system having multi-chamber compartments, and in U.S. Pat. No. 4,475,196, which discloses an osmotic drug delivery system. Many other such implants, delivery systems, and modules are known to those skilled in the art.

In certain embodiments, the antibodies and/or immunoconjugates described herein can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade (1989) J. Clin. Pharmacol. 29: 685). Illustrative targeting moieties include, but are not limited to folate or biotin (see, e.g., U.S. Pat. No. 5,416,016); mannosides (Umezawa et al., (1988) Biochem. Biophys. Res. Commun. 153: 1038); antibodies (Bloeman et al. (1995) FEBS Lett. 357:140; Owais et al. (1995) Antimicrob. Agents Chemother. 39:180); surfactant protein A receptor (Briscoe et al. (1995) Am. J. Physiol. 1233:134).

### Kits.

Where a radioactive, or other, effector is used as a diagnostic and/or therapeutic agent, it is frequently impossible to put the ready-for-use composition at the disposal of the user, because of the often poor shelf life of the radiolabeled compound and/or the short half-life of the radionuclide used. In such cases the user can carry out the labeling reaction with the radionuclide in the clinical hospital, physician's office, or laboratory. For this purpose, or other purposes, the various reaction ingredients can then be offered to the user in the form of a so-called “kit”. The kit is preferably designed so that the manipulations necessary to perform the desired reaction should be as simple as possible to enable the user to prepare from the kit the desired composition by using the facilities that are at his disposal. Therefore the invention also relates to a kit for preparing a composition according to this invention.

In certain embodiments, such a kit comprises one or more antibodies or immumoconjugates described herein. The antibodies or immumoconjugates can be provided, if desired, with inert pharmaceutically acceptable carrier and/or formulating agents and/or adjuvants is/are added. In addition, the kit optionally includes a solution of a salt or chelate of a suitable radionuclide (or other active agent), and (iii) instructions for use with a prescription for administering and/or reacting the ingredients present in the kit.

The kit to be supplied to the user may also comprise the ingredient(s) defined above, together with instructions for use, whereas the solution of a salt or chelate of the radionuclide, defined sub (ii) above, which solution has a limited shelf life, may be put to the disposal of the user separately.

The kit can optionally, additionally comprise a reducing agent and/or, if desired, a chelator, and/or instructions for use of the composition and/or a prescription for reacting the ingredients of the kit to form the desired product(s). If desired, the ingredients of the kit may be combined, provided they are compatible.

In certain embodiments, the immunoconjugate can simply be produced by combining the components in a neutral medium and causing them to react. For that purpose the effector may be presented to the antibody, for example, in the form of a chelate.

When kit constituent(s) are used as component(s) for pharmaceutical administration (e.g. as an injection liquid) they are preferably sterile. When the constituent(s) are provided in a dry state, the user should preferably use a sterile physiological saline solution as a solvent. If desired, the constituent(s) may be stabilized in the conventional manner with suitable stabilizers, for example, ascorbic acid, gentisic acid or salts of these acids, or they may comprise other auxiliary agents, for example, fillers, such as glucose, lactose, mannitol, and the like.

While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Creation of Targeted Interferon Gamma Capable of Forming IFNγ Dimers

Given the requirement for dimer formation for IFNγ activity, we examined the use of several different linkers for their ability to allow functional dimer formation by two IFNγ moieties joined to the carboxy terminus of the IgG heavy chain. One challenge is to make sure that linkers are long enough so that different domains do not impair each other in biologic activity. Additionally, there must not be cleavage of the linkers. Linkers that we examined included LTEEQQEGGG (SEQ ID NO:1) and LTEEQQEGGGIFNγTEEQQEGGG (SEQ ID NO:2) based on a report of a biologically active single-chain version of IFNγ (Landar et al. (2000) J. Mol. Biol. 299: 169), LAKLKQKTEQLQDRIAGGG (SEQ ID NO:3) chosen from a linker database, the hinge of IgG3 (LELKTPLGDTTHTCP RCPEPKSCDTPPPCP RCPEPKSCDTPPPCP RCPEPKSCDTPPPCP RCPGG, SEQ ID NO:4), the hinge of IgG3 lacking cys (LELKTPLGDTTHTSP RSPEPKSSDTPPPSP RSPEPKSSDTPPPSP RSPEPKSSDTPPPSP RSPGG, SEQ ID NO:5), and the hinge of IgG1 lacking cys (LEPKSSDKTHTSPPSPSGG) (Table 1). The linkers have different properties. IgG1 delta cys and 1qo0E_1 are similar in length (18 and 19 aa, respectively) but have different conformations (coil and alpha helix, respectively). IgG3 and IgG3 delta cys are of the same length but there are no disulfide bonds in the latter. The Landar linker has a relatively short 10 aa sequence.

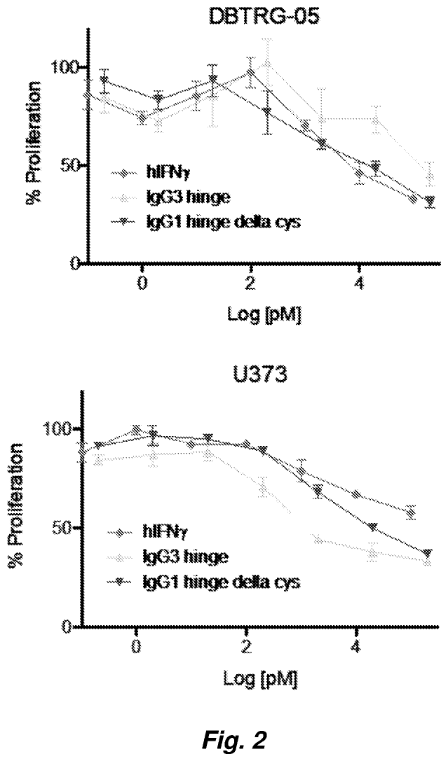

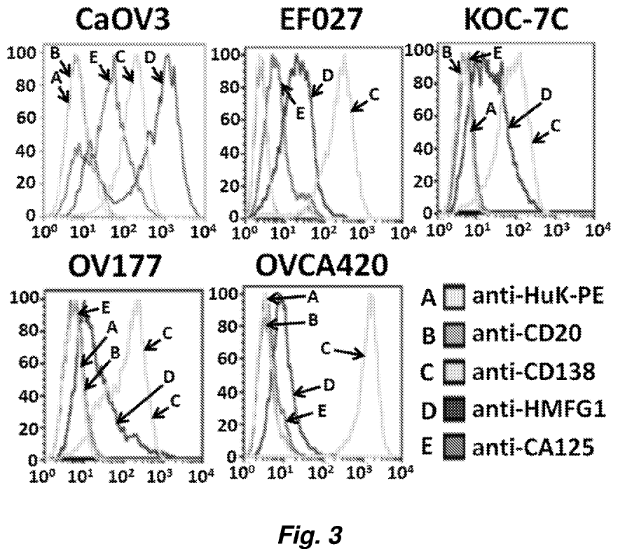

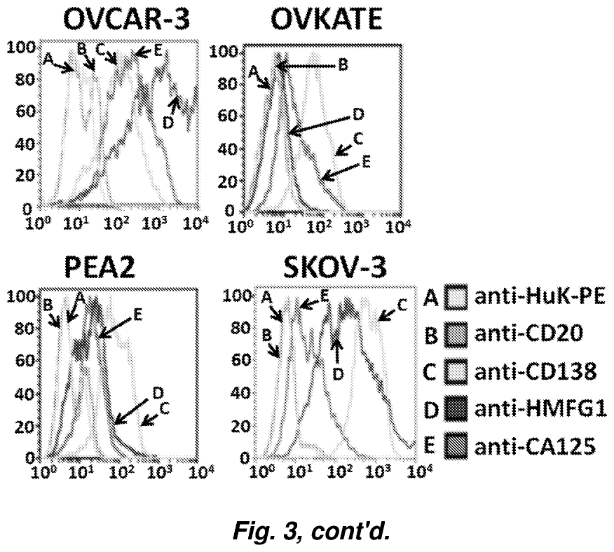

IFNγ was joined with the different linkers after the C_{H}3 domain of anti-CSPG4, transiently expressed in 293T cells and protein isolated from culture supernatants using protein A Sepharose. The fusion proteins were then evaluated for their ability to inhibit the proliferation of OVCAR3, an ovarian cancer, T98MG, a glioma, and A375, a melanoma (FIG. 1). OVCAR3 and T98MG do not express CSPG-4, while A375 expresses it to high levels. Thus for OVCAR3 and T98MG we are examining the relative efficacy of untargeted fusion protein in comparison to untargeted recombinant IFNγ; for A375 we are comparing anti-CSPG-4 targeted IFNγ with untargeted recombinant IFNγ. For OVCAR3, the most effective IFNγ fusion utilized the IgG3 hinge with the second most effective using the IgG1 hinge 4 cys. Interestingly, both were more effective than this preparation of recombinant IFNγ. For T98MG, the most effective fusion protein contained the 1qo0E_1 linker followed closely by the fusion proteins with the IgG1 hinge 4 cys and the IgG3 hinge which showed activity similar to that of recombinant IFNγ. When targeted by anti-CSPG-4, fusion proteins containing the IgG3 hinge, IgG1 hinge 4 cys linker and the 1qo0E_1 linker showed similar activity. All three were several orders of magnitude more effective than recombinant hIFNγ. Comparison of untargeted fusion proteins in 2 additional glioblastoma cell lines that did not express CSPG-4 (DBTRG-05 and U373), showed that the fusion with the IgG1 hinge 4 cys was more active in one (DBTRG-05), while the fusion with the IgG3 hinge was more active in the other (U373) (FIG. 2).

Based on our results, initially we elected to use fusions with the IgG3 hinge and made stable transfectants of CHO cells expressing anti-CD138-IgG3 hinge-IFNγ. However, we found that there was significant cleavage of the protein at the site of the linker during synthesis (data not shown). Therefore, we made stable transfects of CHO expressing anti-CD138-IgG1 hinge Δ cys-IFNγ. With this linker the IFNγ is active and the resulting fusion proteins are stable.

### Relative Efficacy of Different Fusion Proteins Against Ovarian Cancer Cell Lines

A screen of 36 different ovarian cancer cell lines by Dr. Dennis Slamon showed differential sensitivity to type I (IFNα and IFNβ) and type II (IFNγ) IFNs. From this panel we chose nine cell lines with differing sensitivities to IFN for analysis. These represented different cell types including two adenocarinomas (CaOV-3 and PEA2), one mucinous (EFO27), two clear cells (KOC-7C) and four serous (OV177, OVCA420, OVCAR3 and OVKATE).

Since one goal is to be able to target IFNs specifically to ovarian cancer cells, we assessed the cell lines for their surface expression of different potential antigens including CD20, CD138, HMFG1 (MUC1) and CA125. Although HMFG1 and CA125 are more traditional ovarian cancer antigens, we found that CD138 was most consistently expressed. CD20 was not expressed by any of the cell lines and can serve as a negative control of targeting.

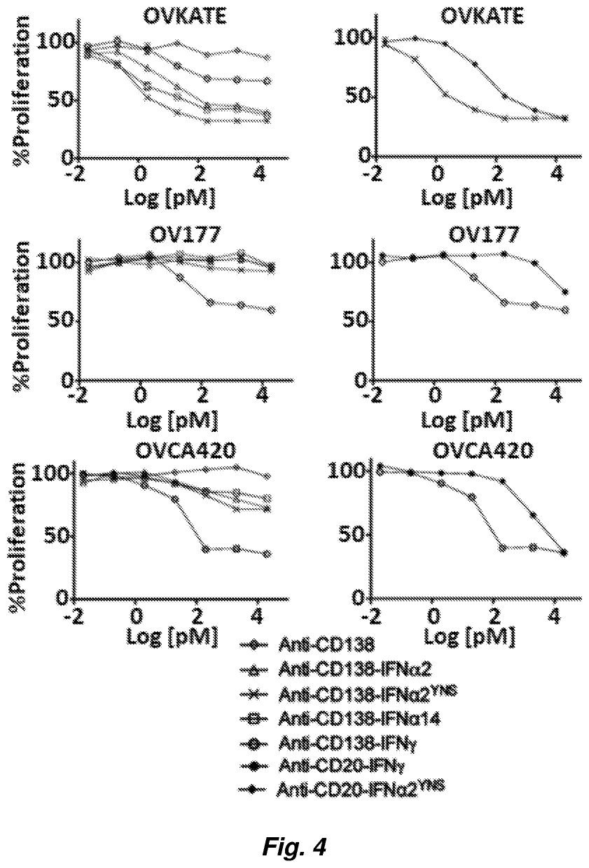

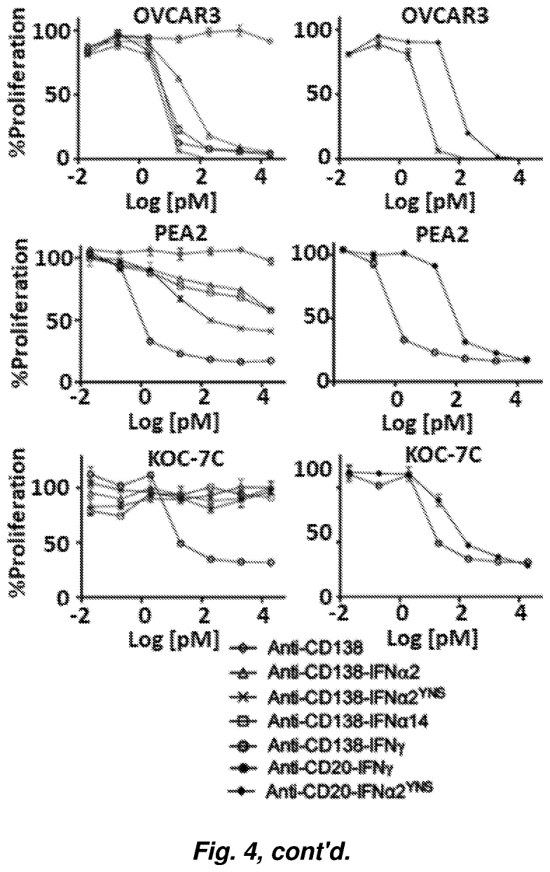

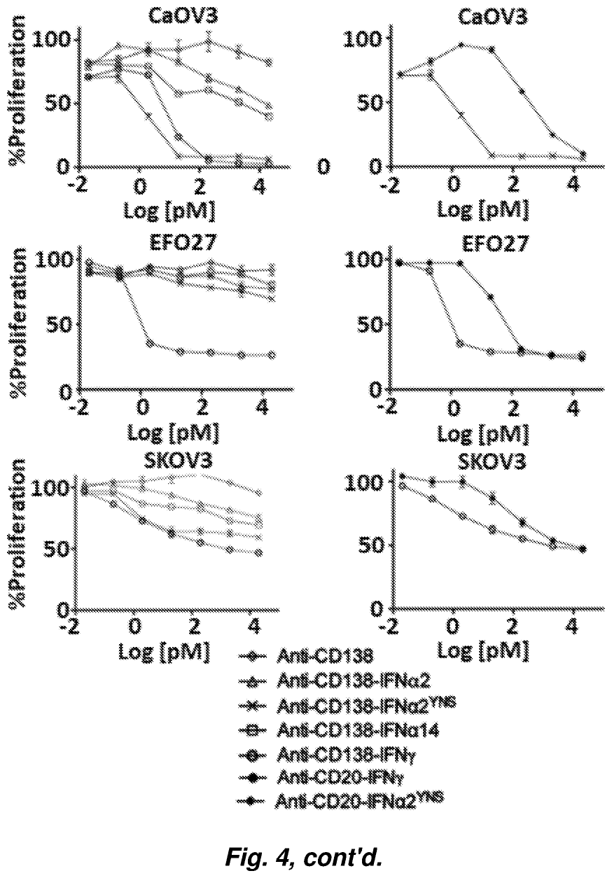

Fusion proteins were constructed with four different IFNs: IFNs 2, IFNα2^{YNS}, IFNα14, and IFNγ. IFNα2 was chosen for our initial fusion proteins since it is the IFN most frequently used in the clinic. However, IFNα14 has more potent cytotoxic activity than IFNα2 and we also made a fusion protein containing IFNα14. All type I IFNs are recognized by a single shared receptor composed of two transmembrane proteins, IFNAR1 and IFNAR2. However, IFNβ has stronger receptor binding and more potent anti-proliferative and pro-apoptotic activities than IFNγ against many cancers. Unfortunately fusion of human IFNβ to the heavy chain of IgG severely compromises its efficacy (unpublished data). As an alternative approach we made a fusion protein containing a mutant IFNα2 (IFNα2^{YNS}) that binds IFNAR1 with a 30-fold higher affinity and exhibits increased anti-proliferative activity that is similar to that of IFNβ. The anti-proliferative activity of the fusion proteins was evaluated using the panel of 9 ovarian cell lines. Anti-CD138 served as a control for inhibition of proliferation in the absence of an attached IFN. The ovarian cancer cell lines do not express CD20 and fusions containing anti-CD20 were used to evaluate the contribution of specific targeting to the inhibition of proliferation. The results are shown in FIG. 4.

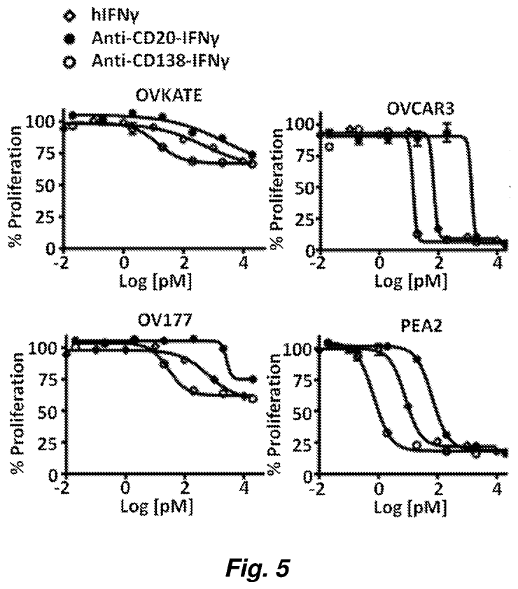

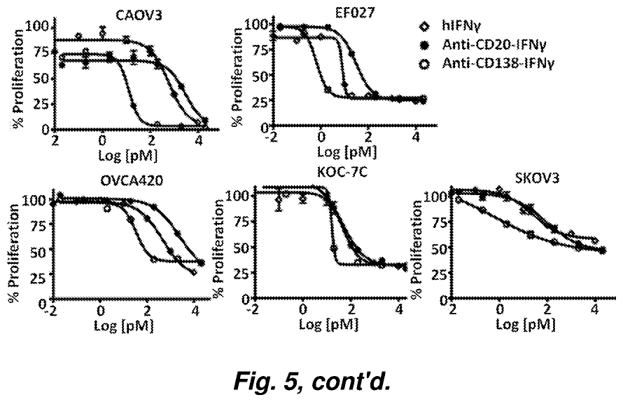

For all cell lines anti-CD138 lacking an attached IFN did not exhibit anti-proliferative activity. For OVKATE and CaOV3 the fusion protein containing IFNα2^{YNS} showed superior anti-proliferative activity. For OVCAR3 fusions with IFNα2^{YNS}, IFNα14, and IFNγ showed similar activity which was superior to that of the fusion protein containing IFNα2. However, for the other 6 cell lines the fusion protein containing IFNγ showed superior anti-proliferative activity. For all cell lines greater inhibition of proliferation was seen with the targeted IFN fusion protein than with a fusion protein containing the same IFN but directed to a surface antigen not expressed by the ovarian cell line. Untargeted fusion protein was less effective than recombinant hIFNγ in inhibiting the proliferation of most ovarian cancer cell lines (FIG. 5). However, targeting improved the efficacy and for all cell lines targeted fusion protein was more effective than recombinant hIFNγ.

### Relative Efficacy of Different Fusion Proteins Against the Glioblastoma T98MG

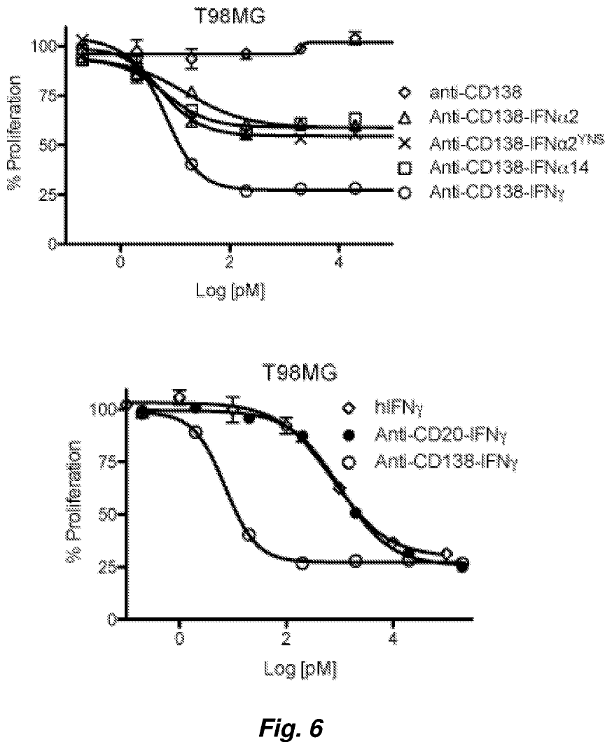

To determine the relative efficacy of the fusion proteins against a different cancer, the glioblastoma T98MG was incubated with the indicated proteins for 6 days and the amount of metabolically active cells remaining determined by MTS assay. Anti-CD138-IFNγ was more effective than any of the IFNα fusion proteins (FIG. 6, top panel). T98MG expresses CD138 but not CD20 and targeted fusion protein was approximately 3 logs more effective than untargeted fusion protein (FIG. 6, bottom panel). Untargeted fusion protein had anti-proliferative activity comparable to recombinant hIFNγ.

### Efficacy of IFNγ Fusions Against Multiple Myeloma

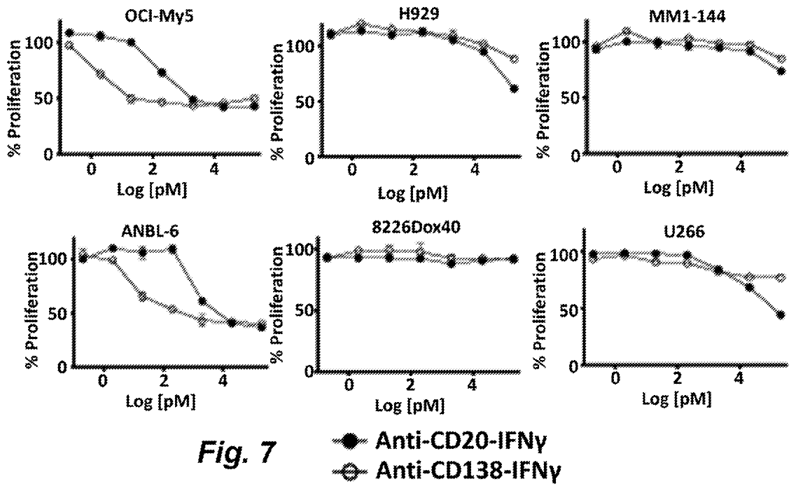

Multiple myeloma expresses CD138 but not CD20. The myeloma cell lines H929, MM1-144, 8226Dox40 and U266 showed little to no sensitivity to inhibition of proliferation by IFNγ at the concentrations tested (FIG. 7). In contrast, both OCI-My5 and ANBL-6 showed sensitivity to IFNγ which was increased approximately 2 logs through targeting.

Fusions with Mouse IFNγ

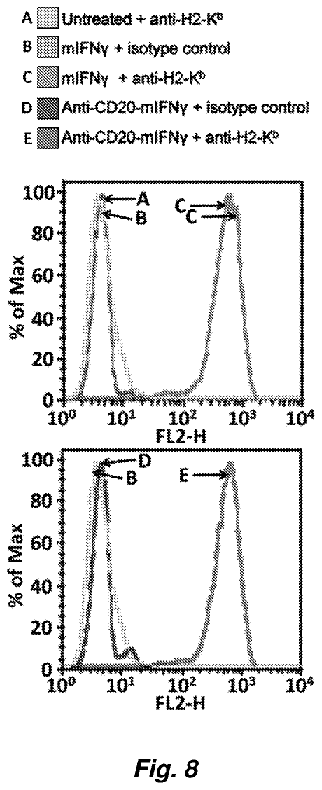

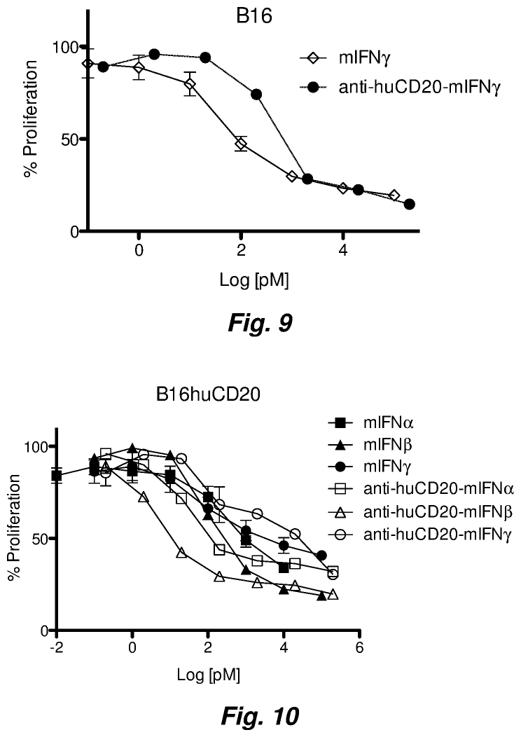

Mouse IFNγ was fused to the carboxy terminus of mouse IgG2a using the IgG1 hingeΔcys linker that was used to create fusions with human IFNγ. To determine if the fusion was active, B16 cells were incubated for 24 hours with either 1.5 nM mouse IFNγ (mIFNγ) or 0.75 nM anti-CD20-mIFNγ (B16 does not express CD20) and the expression level of H2-K^{b} determined by flow cytometry (FIG. 8). The fusion protein and recombinant mIFNγ upregulated the expression of class 1 MHC to the same extent. However, when the anti-proliferative activity against B16 of mIFNγ and anti-CD20-mIFNγ were compared, mIFNγ had greater activity than anti-CD20-mIFNγ (FIG. 9).

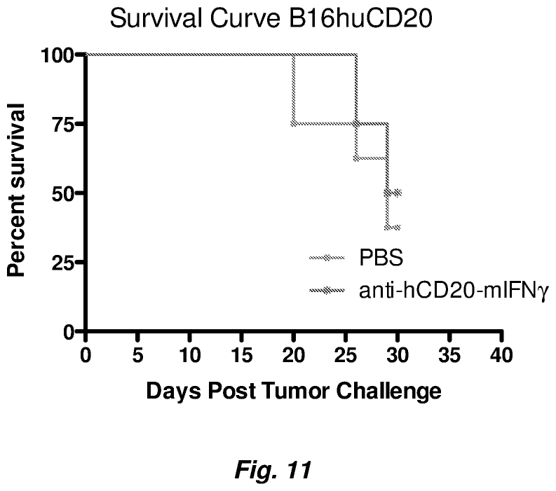

Analysis of B16huCD20, which expresses human CD20, showed that the cell line was less sensitive to mIFNγ than to the type I IFNs (FIG. 10). Targeted anti-huCD20-mIFNγ displayed similar activity to recombinant mIFNγ. Targeted anti-hCD20-mIFNγ was not protective against tumor growth at the concentration tested (FIG. 11).

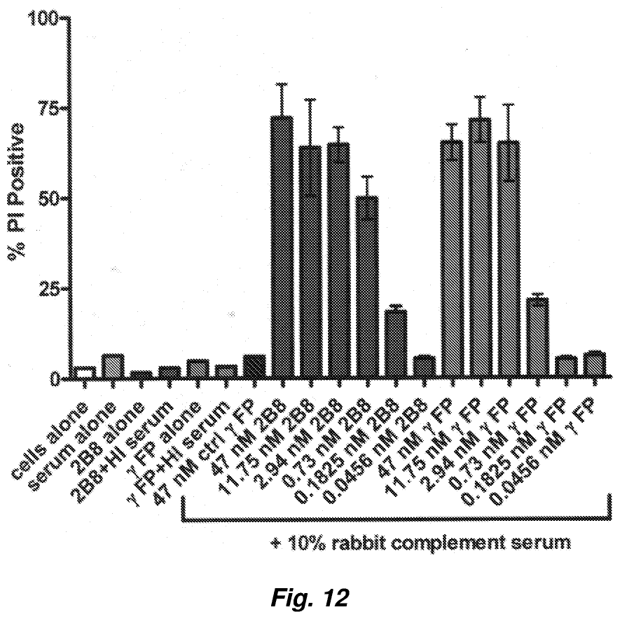

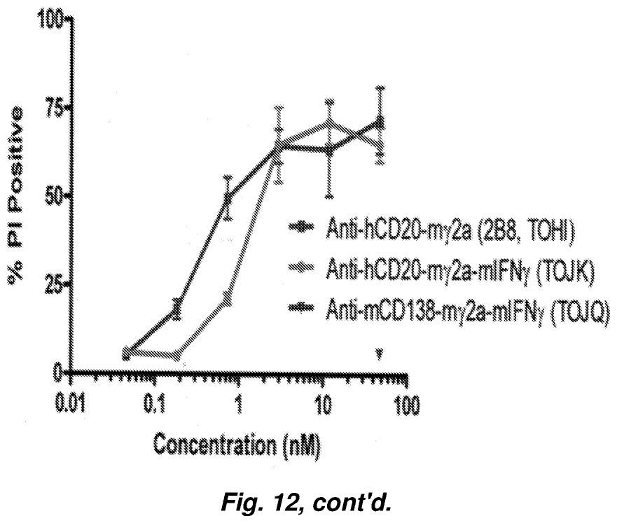

The 38C13 lymphoma was used to further analyze the properties of the fusions containing mIFNγ. The fusion protein was found to be somewhat less active in its ability to activate complement mediated cytolysis (FIG. 12).

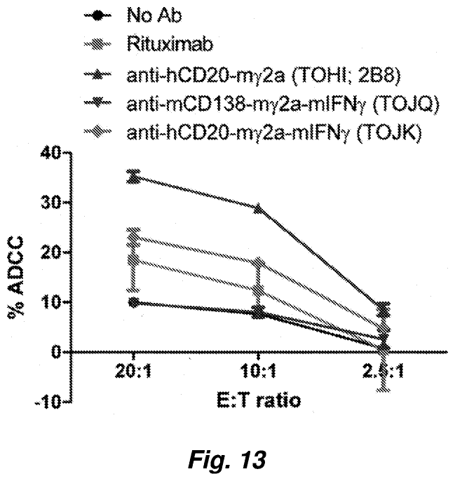

The fusion protein was able to carry about ADCC, but was less effective than the targeted anti-huCD20 with the murine IgG2a constant region which was more effective than Rituximab. (FIG. 13).

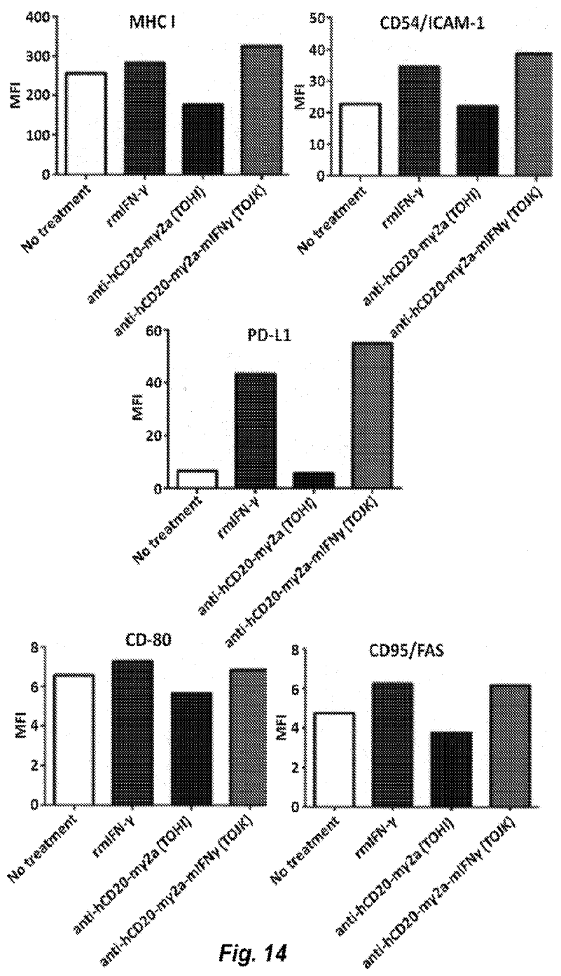

The effects of mIFNγ, anti-hCD20, and anti-hCD20-mIFNγ on the expression of MHCI, ICAM-1, PD-L1, FAS and CD80 by 38C13-huCD20 following 24 hours of treatment were tested (FIG. 14). Both mIFNγ and anti-hCD20-IFNγ slightly upregulated the expression of MHC I, FAS and CD80 while anti-hCD20 downregulated their expression. Both IFNγ and anti-hCD20-mIFNγ strongly upregulated the expression of ICAM1 and PD-L1 while anti-hCD20 had little effect.

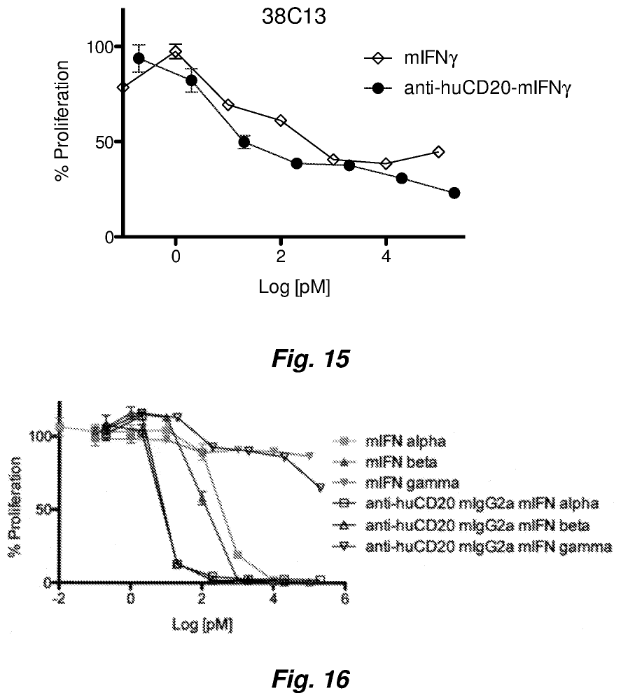

Untargeted anti-huCD20-mIFNγ was found to be slightly more effective than mIFNγ in inhibiting the growth of 38C13 (FIG. 15). However, neither mIFNγ nor antiCD20-mIFNγ were effective in inhibiting the growth of 38C13huCD20 in vitro (FIG. 16).

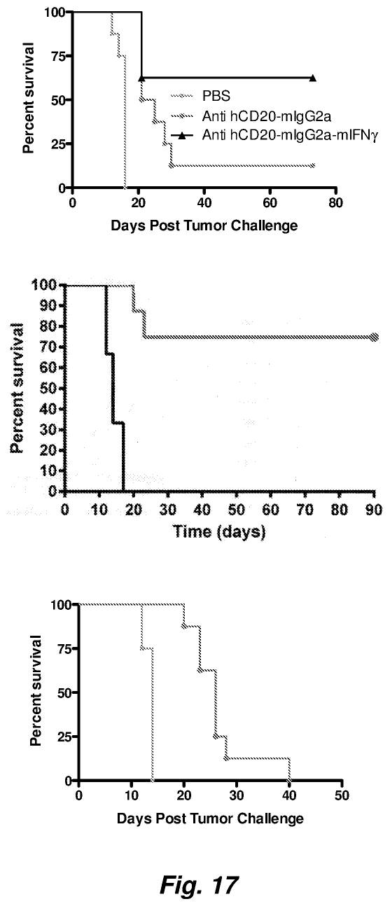

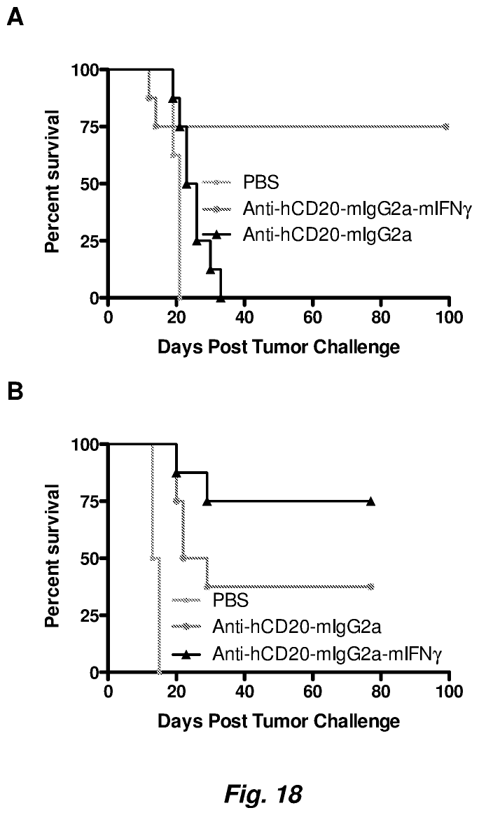

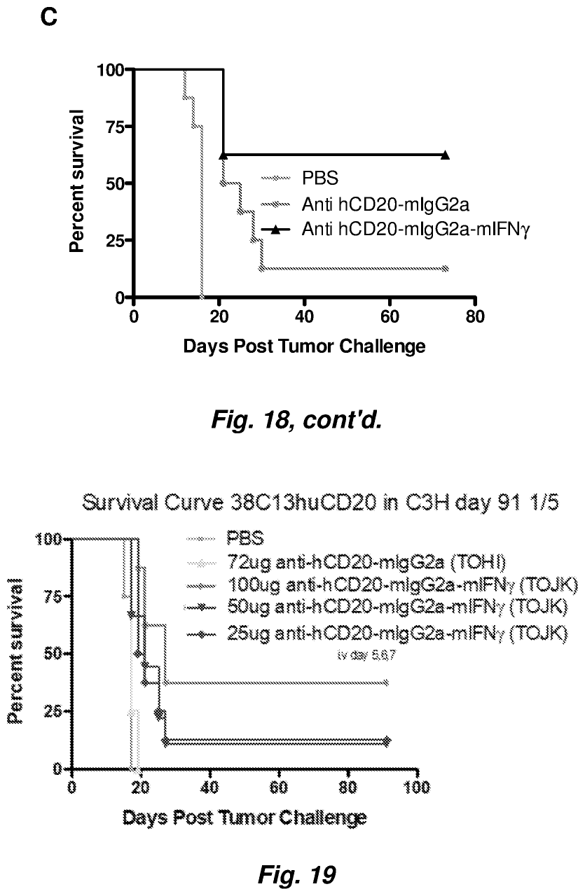

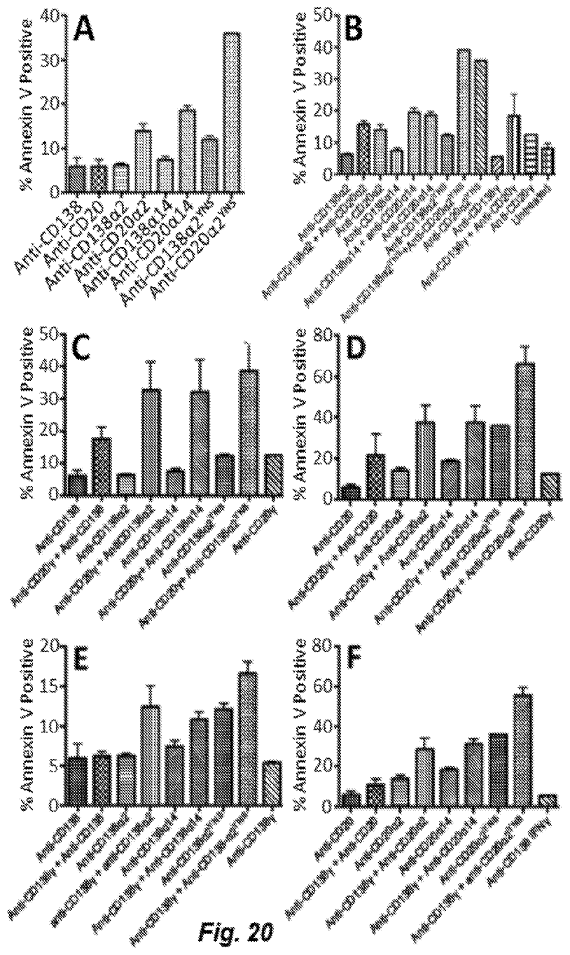

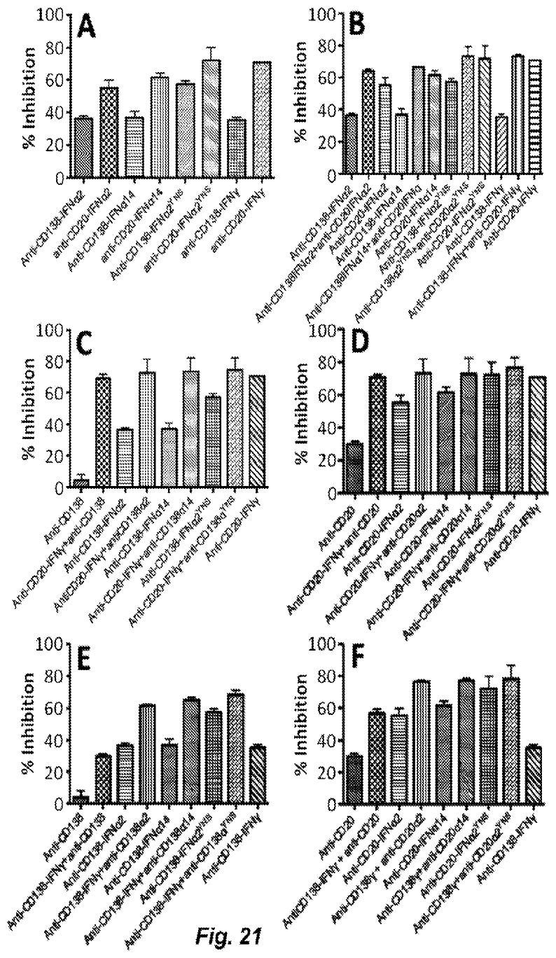

Unexpectedly, although not effective in vitro, anti-hCD20mIgG2a-mIFNγ was very effective in inhibiting tumor growth in vivo (FIG. 17) and was more effective than anti-hCD20mIgG2a (FIG. 18).

Higher doses of anti-hCD20mIgG2a-mIFNγ were found to be more effective in inhibiting tumor growth in vivo (data not shown).

### Activity of Fusion Proteins Against Waldenstrom Macroglobulinemia (WM)

The MWCL-1 cell line was used for these studies. The cell line was established from a bone marrow aspirate of a 73-year-old male patient with IgM-κ WM. It contains the EBV gene product EBNA1, but does not appear to be actively replicating virus and was reported to be both CD20 and CD138 positive.

We confirmed that all cells were positive for the expression of both CD138 and CD20, with brighter staining seen using anti-CD20.

### Ability of Proteins Treatment to Induce Apoptosis:

To evaluate the ability of the single fusion proteins to induce apoptosis in MWCL-1, cells were treated with 12.5 nM of the indicated proteins for 5 days and apoptosis evaluated by flow-cytometry following staining with Annexin V and PI. Overall fusion proteins targeting CD20 were more potent than those with the same IFN targeting CD138, consistent with the apparently greater expression of CD20 by the cells (FIG. 20, panel A). Anti-CD20α2^{YNS} was the most potent, followed by anti-CD20α14 with anti-CD20-IFNγ and anti-CD20-IFNα2 being the least potent. Using the fusion proteins targeting CD138, only anti-CD138α2^{YNS} was effective. When fusion proteins were used that simultaneously targeted CD20 and CD138, the activity was primarily contributed by the anti-CD20 fusion protein with the additional targeting to CD20 contributing little to increasing the amount of apoptosis induced (FIG. 20, panel B).

### Simultaneous Targeting of Type I and Type II IFN Enhances the Induction of Apoptosis:

When anti-CD20-IFNγ was targeted along with either anti-CD138 or anti-CD20 unfused or fused to type IFN, increased induction of apoptosis was seen in all combinations (FIG. 20, panels C and D). However, more effective induction of apoptosis was seen using the anti-CD20 proteins (compare FIG. 20, panel C and panel D). The most effective combination was anti-CD20-IFNγ with anti-CD20α2^{YNS}, in which approximately 60% of the cells were Annexin V positive following treatment.

Anti-CD138-IFNγ did not result in increased induction of apoptosis when used in combination with anti-CD138 or anti-CD20. However, it did result in increased apoptosis when used in combination with any of the fusions of anti-CD138 and anti-CD20 with type I interferon. More effective induction of apoptosis was seen when anti-CD138-IFNγ was combined with anti-CD20 proteins (compare FIG. 20, panels E and F). The most effective combination was anti-CD138-IFNγ+anti-CD20α2^{YNS}.

### Ability of Proteins to Inhibit Proliferation:

To determine the ability of the proteins to inhibit proliferation, MWCL-1 cells were incubated with 12.5 nM protein for 72 hours, then pulsed with ³H-thymidine for 8 hours and proliferation measured by determining the amount of ³H-thymidine incorporated into DNA. Proteins targeting CD20 were more effective in inhibiting proliferation than those targeting CD138, with anti-CD20-IFNγ and anti-CD20-IFNα2^{YNS} most effective (FIG. 21, panel A). Simultaneously targeting the same IFN by treating with anti-CD20-IFN+anti-CD138-IFN did little to increase the inhibition of proliferations seen using only anti-CD20-IFN (FIG. 21, panel B).

Anti-CD20-IFNγ was so effective in inhibiting proliferation that the addition of an anti-CD138- or anti-CD20-type I IFN did little to increase the inhibition of proliferation observed (FIG. 21, panels C and D). When anti-CD138-IFNγ was combined with anti-CD138 and anti-CD20 both unfused and fused with type I IFN, enhanced inhibition of proliferation was observed; however for both anti-CD138-IFNα2^{YNS} and anti-CD20IFNα2^{YNS}, the enhancement was minimal since the fusions with IFNα2^{YNS} were so effective when used by themselves (FIG. 21, panels E and F).

### Example 2

### STAT Activation Following Fusion Protein Treatment

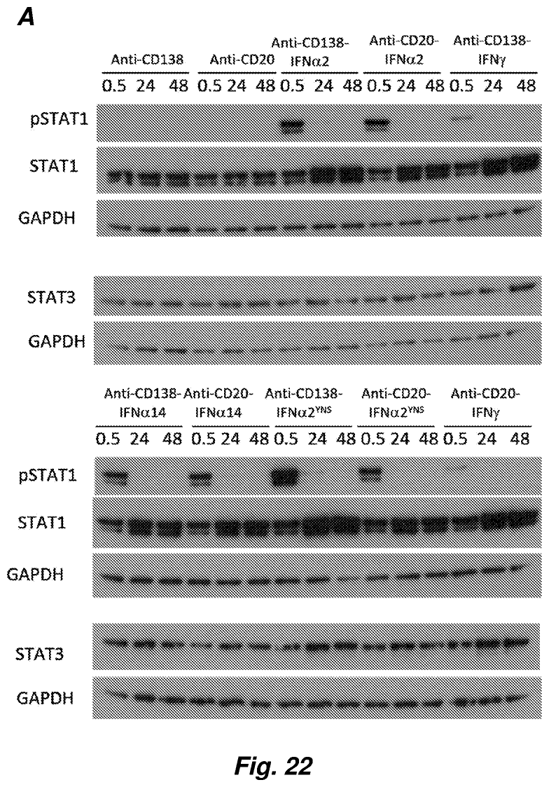

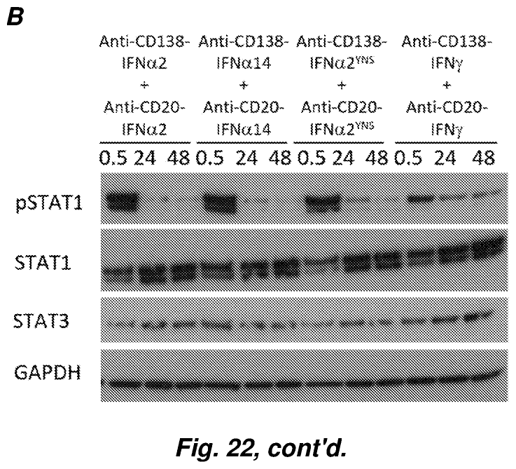

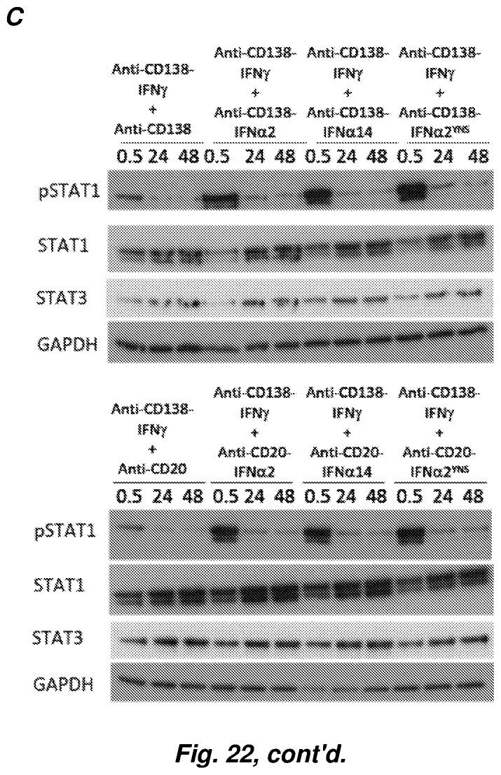

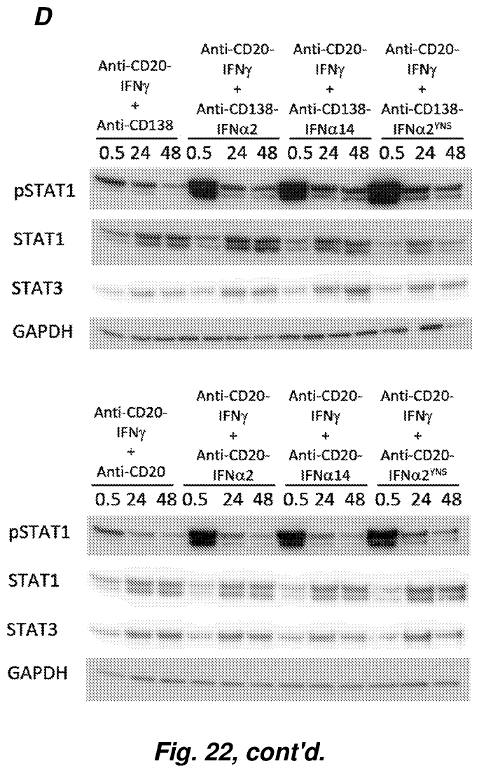

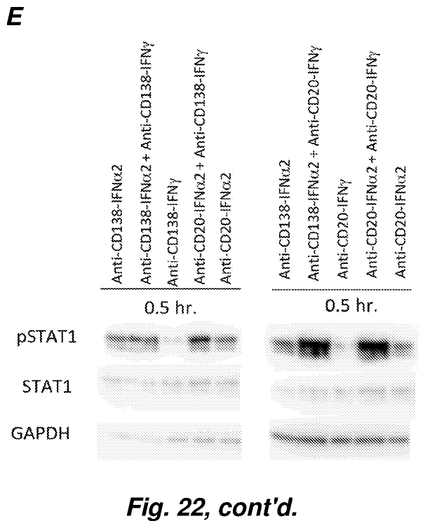

STAT activation is one of the first events following treatment with interferon. To assess STAT activation following treatment with the different proteins, MWCL1 cells were incubated with 12.5 nM of the indicated proteins for 0.5, 24 or 48 hours, extracts made and assayed by Western blot for pSTAT1, total STAT1, pSTAT3 and total STAT3 (FIG. 22, panel A). Treatment with anti-CD138 or anti-CD20 did not result in STAT activation. Treatment with both anti-CD20 and anti-CD138 fusions with IFN-o 2, IFNα2^{YNS} and IFNα14 resulted in robust phosphorylation of STAT1 at 0.5 hours which was not longer evident at 24 hours. Treatment with anti-CD20 and anti-CD138 fused to IFN-γ interferon resulted in some phosphorylation of STATI at 0.5 hours, but this was not as robust as the phosphorylation seen following treatment with fusions with type I IFNs. However, treatment with fusion with both type I and type II IFNs resulted in strong upregulation of STATI protein levels which was seen at 24 and 48 hours. No phosphorylation of STAT3 was seen following any treatments.

Simultaneous targeting of the same interferon species to both CD20 and CD138 resulted in strong phosphorylation of STATI when type I IFNs were used; this phosphorylation persisted and could still be seen 48 hours following treatment (STAT FIG. 22, panel B). Phosphorylation following treatment with the IFNγ fusion proteins also was evident at 24 and 48 hours following treatment when both CD20 and CD138 were simultaneously targeted. However, when extracts from cells treated for 0.5 hours with anti-CD20IFNα2, anti-CD138-IFNα2 or anti-CD20IFNα2+anti-CD138-IFNα2 were run side-by-side on the same gel, no enhanced STAT1 phosphorylation was seen when both proteins were targeted. The same was true when extracts from cells treated for 0.5 hours with anti-CD20-IFNγ, anti-CD138-IFNγ or anti-CD20-IFNγ+anti-CD138-IFNγ were compared. As had been seen earlier, increased levels of STAT1 protein were seen at 24 and 28 hours following all fusion protein treatments and no pSTAT3 was seen.

To address the question of whether simultaneous targeting of type I and type II interferon would influence the level of STAT activation observed, MWCL1 cells were incubated with 12.5 nM of anti-CD138-IFNγ and proteins targeting CD138 or CD20 either unfused or fused to type I IFN (FIG. 22, panel C) or with 12.5 nM of anti-CD20-IFNγ and proteins targeting CD138 or CD20 either unfused or fused to type I IFN (FIG. 22, panel D). Combining anti-CD138-IFNγ with anti-CD138 or anti-CD20 resulted in little increase in STAT1 phosphorylation over what was observed with only anti-CD138-IFNγ treatment. However, combining anti-CD138-IFNγ with fusions with type I IFN resulted in phosphorylation of STAT1 that could still be observed at 48 hours following treatment (FIG. 22, panel C). In addition, side-by-side comparisons showed that more robust STAT I phosphorylation was seen when anti-CD138-IFNγ was combined with either anti-CD138-IFNα2 or anti-CD20-IFNα2 than with any of the single treatments (FIG. 22, panel E). Although no phosphorylation of STAT3 was observed increased levels of STAT3 were observed at 24 and 48 hours.

When MWCL1 cells were incubated with 12.5 nM of anti-CD20-IFNγ and proteins targeting CD138 or CD20 a small increase in STAT1 phosphorylation was observed compared with only anti-CD20-IFNγ treatment; notable was the fact that pSTAT1 was observed at 24 and 48 hours following this treatment. Moreover, combining anti-CD20-IFNγ with fusions with type I IFN resulted in very robust phosphorylation of STAT1 that could still be observed at 48 hours following treatment (FIG. 22, panel D). In addition, side-by-side comparisons showed that more robust STAT I phosphorylation was seen when anti-CD20-IFNγ was combined with either anti-CD138-IFNα2 or anti-CD20-IFNα2 than with any of the single treatments (FIG. 22, panel E). Again no phosphorylation of STAT3 was observed. However, an increase in STAT3 protein levels at 24 and 48 hours was observed following all treatments (FIG. 22, panel D).

Extracts were also blotted for STATS and pSTAT5. No phosphorylation of STATS was seen

### In Vivo Protection

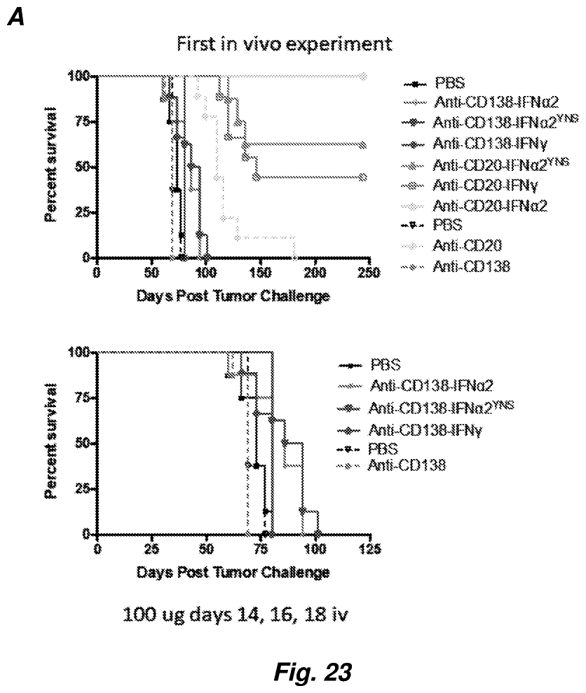

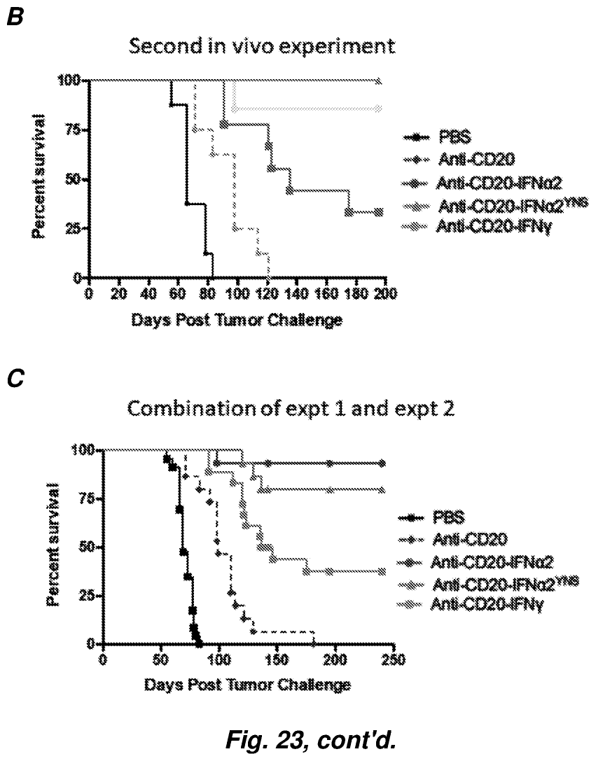

To investigate in vivo protection, SCID mice were injected subcutaneously with 1×10⁷ WMCL1 cells. On days 14, 16 and 18 following tumor implantation at a time when tumors were palpable mice were treated i.v. with 100 μg of the indicated proteins and monitored for tumor growth. Mice were euthanized when tumors were 1.5 cm in size (FIG. 23, panel A). For the initial experiment, all treatments were performed at the same time except anti-CD20, anti-CD138 and one PBS treatment which were done at a separate time and are plotted as dashed lines. Proteins targeting CD20 are much more protective than those targeting CD138. Even anti-CD20 that was not fused to interferon gave significant protection (p<0.0001 compared to PBS). Among the proteins targeting CD20, anti-CD20-IFNα2 was most protective, preventing tumor growth in all mice. Anti-CD20-IFNγ and anti-CD20-IFNα2^{YNS} showed similar levels of protection, with 4 of 9 and 5 of 8 mice respectively remaining tumor free. At the end of the experiment (250 days) the sera from the surviving mice were analyzed by ELISA and found to not contain any IgM indicating that the mice were truly tumor free. The experiment was repeated (FIG. 23, panel B) and similar results were obtained. Treatment with anti-CD20 provided some protection. Fusions with type I IFN were more protective than fusions with IFNγ; however in this case anti-CD20-IFNα2^{YNS} protected more mice than did anti-CD20-IFNα2.

Analysis of the results obtained with proteins that target CD138 showed that the median survival time of mice treated with PBS was 71 days while the median survival of mice treated with anti-CD138 was 69 days, so unlike what was observed with anti-CD20 no survival benefit is seen with the unfused protein. A small survival benefit was seen with the anti-CD138-IFN fusion proteins: the median survival time following treatment with anti-CD138-IFNα was 86 days (p=0.001), following treatment with anti-CD138-IFNα2^{YNS} it was 90 days (p=0.0003), and following treatment with anti-CD138-IFNγ it was 80 days (p=0.04).

Analysis of the results of the combined experiments showed that anti-CD20 provides significant protection (p<0.0001 compared with PBS) (see, e.g., FIG. 23, panel C). However both anti-CD20-IFNγ and anti-CD20-IFNα2^{YNS} are significantly more protective than anti-CD20 (p=0.004 and <0.0001 respectively). Anti-CD20-IFNα2^{YNS} is significantly more protective than anti-CD20-IFNγ (p=0.02). However there is no significant difference in the protection provided by the two type I IFN fusion proteins (p=0.34).

Anti-Proliferative Activity of IFN-7 Fusion Proteins in Combination with Bortezomib or Ibrutinib

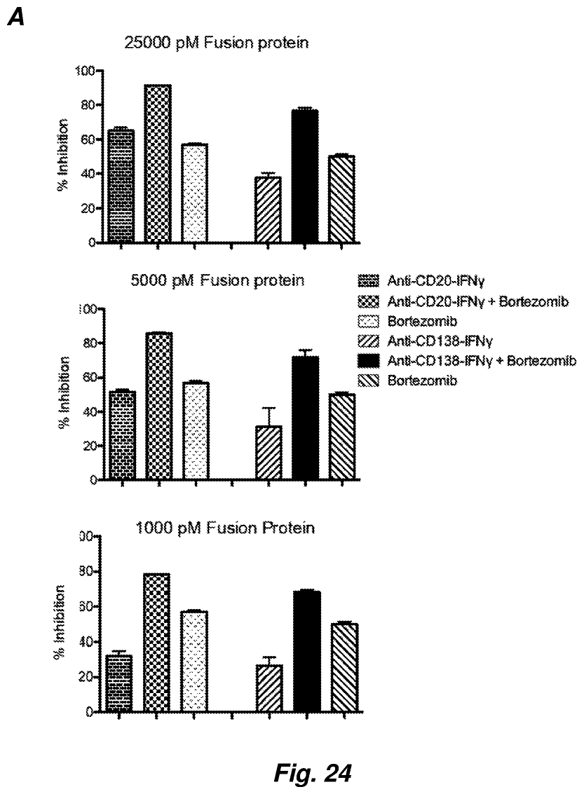

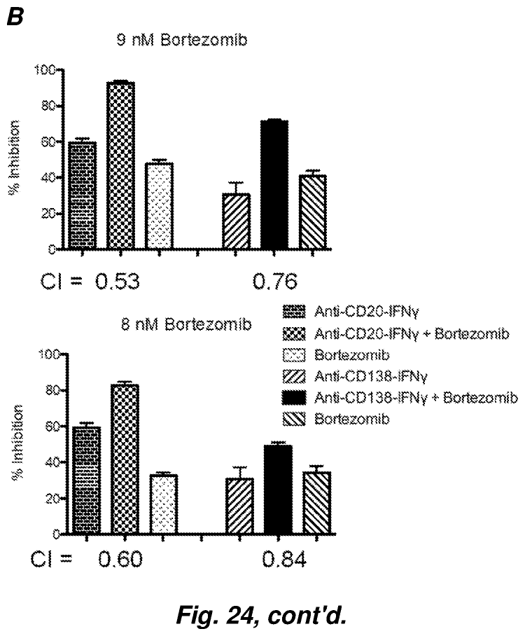

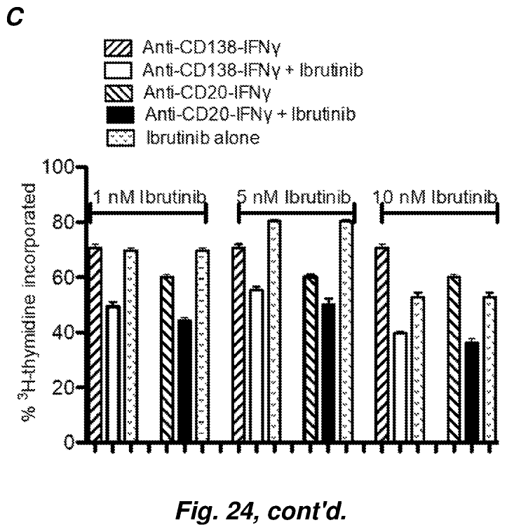

Although bortezomib showed dose-dependent inhibition of proliferation, no enhanced inhibition of proliferation as indicated by decreased incorporation of ³H-thymidine was seen when MWCL-1 cells were incubated with bortezomib and fusions with type I interferon (Data not shown). In contrast, increased inhibition of proliferation was seen when MWCL-1 cells were incubated different concentrations of anti-CD20-IFNγ and anti-CD138-IFNγ fusion proteins and bortezomib for 72 hours. In the initial experiment the bortezomib concentration was held constant at 8 nM and cells were incubated with different concentrations of fusion protein (FIG. 24, panel A). Increased inhibition of proliferation was seen for both concentrations of fusion protein. In a second experiment cells were incubated with either 9 or 8 nM bortezomib and fusion protein at different concentrations. Shown are the results for 25000 pM of fusion protein with either 9 or 8 nM of bortezomib (FIG. 24, panel B). Using the data from this experiment it was possible to calculate the CI. Although the CI was found it to be less than 1 the results would suggest that the interactions between the fusion proteins and bortezomib are only “moderately” synergistic.

Decreased cell proliferation was also seen following treatment with ibrutinib and fusion proteins containing type II IFN (FIG. 24, panel C). When fusion proteins were used at 200 nM, less proliferation was observed following combination treatment with 1 nM, 5 nM and 10 nM of ibrutinib.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A chimeric construct comprising a full-length immunoglobulin or a camelid antibody attached to an interferon gamma (IFNγ) wherein:
said immunoglobulin or camelid antibody is an antibody that binds to a tumor associated antigen;
a first interferon gamma (IFNγ) is attached to a first constant heavy region 3 (CH₃) of said immunoglobulin or camelid antibody by a first proteolysis resistant peptide linker;
a second interferon gamma is attached to a second constant heavy region 3 (CH₃) of said immunoglobulin or camelid antibody by a second proteolysis resistant peptide linker; and
said first proteolysis resistant linker and said second proteolysis linker have a length and flexibility that permits said first interferon gamma and said second interferon gamma to dimerize.

2. The construct of claim 1, wherein said first proteolysis resistant peptide linker and said second proteolysis peptide linker comprise amino acid sequences independently selected from the amino acid sequences of the peptide linkers shown in Table 1.

3. The construct of claim 2, wherein said first proteolysis resisting peptide linker and/or said second proteolysis resisting peptide linker comprise the amino acid sequence of the Landar linker.

4. The construct of claim 2, wherein said first proteolysis resisting peptide linker and/or said second proteolysis resisting peptide linker comprise the amino acid sequence of the Double landar linker.

5. The construct of claim 2, wherein said first proteolysis resisting peptide linker and/or said second proteolysis resisting peptide linker comprise the amino acid sequence of the 1qo0E_1 linker.

6. The construct of claim 2, wherein said first proteolysis resisting peptide linker and/or said second proteolysis resisting peptide linker comprise the amino acid sequence of the IgG3 hinge linker.

7. The construct of claim 2, wherein said first proteolysis resisting peptide linker and/or said second proteolysis resisting peptide linker comprise the amino acid sequence of the IgG3 hinge delta cys linker.

8. The construct of claim 2, wherein said first proteolysis resisting peptide linker and/or said second proteolysis resisting peptide linker comprise the amino acid sequence of the IgG1 hinge delta cys linker.

9. The construct according to any one of claims 1-8, wherein said interferon gamma comprises a murine interferon gamma, or a truncated and/or mutated murine interferon gamma.

10. The construct of claim 9, wherein said interferon gamma comprises a full-length murine interferon gamma.

11. The construct according to any one of claims 9-10, wherein said murine interferon gamma is not glycosylated.

12. The construct according to any one of claims 9-10, wherein said murine interferon gamma is glycosylated at Asn 38 and/or at ASN 90.

13. The construct according to any one of claims 1-8, wherein said interferon gamma comprises a human interferon gamma or a truncated and/or mutated human interferon gamma.

14. The construct of claim 13, wherein said human interferon gamma is not glycosylated.

15. The construct of claim 13, wherein said human interferon gamma comprises N-linked glycosylation at Asn-25 and/or at Asn-97.

16. The construct according to any one of claims 13-15, wherein said human interferon gamma comprises a full-length human interferon.

17. The construct according to any one of claims 13-15, wherein said human interferon gamma comprises a huIFNγ C-terminally truncated with 1-15 amino acid residues, e.g. with 1, 2, 3, 4, 5, 6, 7, 8, 9, 10, 11, 12, 13, 14, or 15 amino acid residues.

18. The construct according to any one of claims 13-15, and 17, wherein said human interferon gamma comprises a human interferon gamma N-terminally truncated with 1, 2, or 3 amino acid residues.

19. The construct according to any one of claims 13-15, wherein said human interferon gamma comprise a human interferon gamma with an N-terminal addition CYC.

20. The construct according to any one of claims 13-19, wherein said human interferon gamma comprises a cysteine substitutions at one or more of Glu8, Ser70, Ala18, His112, Lys81, Leu121, Gln49, and Leu96 (relative to the amino acid sequence of SEQ ID NO: 13).

21. The construct of claim 20, wherein said human interferon gamma comprises at least one pair of amino acids from predetermined amino acid pairs exchanged for cysteine, wherein said four amino acid pairs are Glu8 and Ser70, Ala18 and His 112, Lys81 and Leu121, and Gln-49 and Leu96.

22. The construct according to any one of claims 1-21, wherein said antibody or camelid antibody preferentially or specifically binds to a tumor associated antigen (TAA) selected from the group consisting of CD138, CSPG4, α-fetoprotein, 5 alpha reductase, 5T4 (or TPBG, trophoblast glycoprotein), AM-1, APC, APRIL, B7 family members, BAGE, Bc12, bcr-abl (b3a2), CA-125, CASP-8/FLICE, Cathepsins, CD1, CD115, CD123, CD13, CD14, CD15, CD19, CD2, CD20, CD200, CD203c, CD21, CD23, CD22, CD38, CD25, CD276, CD3, CD30, CD303, CD33, CD34, CD35, CD37, CD38, CD44, CD45, CD46, CDS, CD52, CD55, CD56, CD59 (791Tgp72), CD7, CD70, CD74, CD79, CDCl₂7, CDK4, CEA, CLL-1, c-MET (or HGFR), c-myc, Cox-2, Cripto, DCC, DcR3, DLL3, E6/E7, EGFR, EMBP, Ena78, endoplasmin, EPCAM, EphA2, EphB3, ETBR, FcRL5, FGF8b and FGF8a, FLK-1/KDR, FOLR1, G250, GAGE-Family, gastrin 17, gastrin-releasing hormone (bombesin), GD2/GD3/GM2, glutathione S-transferase, glycosphingolipid GD2, GnRH, GnTV, gp100/Pme117, gp-100-in4, gp15, gp75/TRP-1, GPNMB, hCG, Heparanase, Her2/neu, Her3, Her4, HLA-DR, HM 1.24, HMB 45, HMTV, HMW-MAA, Hsp70, hTERT (telomerase), IFN-α, IGFR1, IL-13R, iNOS, integrin, Ki 67, KIAA0205, K-ras, H-ras-N-ras, KSA (C017-1A), LDLR-FUT, Leu-M1, Lewis A like carbohydrate, Lewis Y, LIV1, MAGE1, MAGES, Mammaglobin, MAP17, Melan-A/, MART-1, mesothelin, MIC A/B, MN, Mox1, MMP2, MMP3, MMP7, MMP9, MUC16, MUC-1, MUC-2, MUC-3, MUC-4, MUC-16, MUM-1, NaPi2b, Nectin-4, NY-ESO-1, Osteonectin, p15, p16INK4, P170/MDR1, p53, p97/melanotransferrin, PAI-1, PDGF, plasminogen (uPA), PMSA, PRAME, Probasin, Progenipoietin, PSA (phosphatidyl serine antigen), PSM, RAGE-1, Rb, RCAS1, SART-1, SE10, SIRP.alpha., SLAM family members, SLC44A4, SSX gene, family, STAT3, STEAP-1, STn (mucin assoc.), TAG-72, TF (or tissue factor), TGF-α, TGF-θ, Thymosin β15, TNF superfamily members, TPA, TPI, TRP-2, Tyrosinase, VEGF, VLA, ZAG, and β-catenin.

23. The construct of claim 22, wherein said antibody or camelid antibody preferentially or specifically binds to CSPG4.

24. The construct of claim 23, wherein said antibody comprises the CDRs of an antibody selected from the group consisting of 9.2.27, VF1-TP34, VF1-TP34, VF1-TP41.2, TP61.5, 149.53, 149.53, 225.28, 225.28s, 763.74, and scFv-FcC21.

25. The construct of claim 22, wherein said antibody or camelid antibody preferentially or specifically binds to CD138.

26. The construct of claim 25, wherein said antibody comprises the CDRs of an antibody comprises an antibody selected from the group consisting of nBT062, B-B4, BC/B-B4, B-B2, DL-101, 1 D4, MI15, 1.BB.210, 2Q1484, 5F7, 104-9, and 281-2.

27. The construct of claim 22, wherein said antibody or camelid antibody preferentially or specifically binds to a member of the EGF receptor family.

28. The construct of claim 27, wherein said antibody comprises the CDRs of an antibody comprises an antibody selected from the group consisting of C6.5, C6ML3-9, C6MH3-B1, C6-B1D2, F5, HER3.A5, HER3.F4, HER3.H1, HER3.H3, HER3.E12, HER3.B12, EGFR.E12, EGFR.C10, EGFR.B11, EGFR.E8, HER4.B4, HER4.G4, HER4.F4, HER4.A8, HER4.B6, HER4.D4, HER4.D7, HER4.D11, HER4.D12, HER4.E3, HER4.E7, HER4.F8 and HER4.C7.

29. The construct of claim 22, wherein said antibody or camelid antibody preferentially or specifically binds to CD20.

30. The construct of claim 29, wherein said antibody comprises the CDRs of an antibody comprises an antibody selected from the group consisting of rituximab, Ibritumomab tiuxetan, and tositumomab.

31. The construct of claim 22, wherein said antibody or camelid antibody preferentially or specifically binds to endoplasmin.

32. The construct of claim 22, wherein said antibody or camelid antibody preferentially or specifically binds to CD33.

33. The construct of claim 22, wherein said antibody or camelid antibody preferentially or specifically binds to CD276.

34. The construct according to any one of claims 1-33, wherein said antibody or camelid antibody is a full-length immunoglobulin.

35. The construct of claim 34, wherein said antibody is a human antibody.

36. The construct of claim 34, wherein said antibody is a humanized or chimeric antibody.

37. The construct according to any one of claims 1-33, wherein said antibody or camelid antibody is a camelid antibody.

38. A pharmaceutical formulation comprising:
a chimeric construct according to any one of claims 1-37; and
a pharmaceutically acceptable carrier.

39. The pharmaceutical formulation of claim 38, wherein said formulation is a unit dosage formulation.

40. The formulation according to any one of claims 38-39, wherein said formulation is formulated for administration via a route selected from the group consisting of oral administration, nasal administration, rectal administration, intraperitoneal injection, intravascular injection, subcutaneous injection, transcutaneous administration, and intramuscular injection.

41. A method of inhibiting growth and/or proliferation of a cell that expresses or overexpresses CD138, said method comprising contacting said cell with a chimeric construct according to any of claims 1-36, or a formulation according to any one of claims 37-39 in an amount sufficient to inhibit growth or proliferation of said cell.

42. The method of claim 41, wherein said cell is a cancer cell.

43. The method of claim 42, wherein said cancer cell is a metastatic cell.

44. The method of claim 42, wherein said cancer cell is in a solid tumor.

45. The method of claim 42, wherein said cancer cell is cell produced by a cancer selected from the group consisting of multiple myeloma, ovarian carcinoma, cervical cancer, endometrial cancer, kidney carcinoma, gall bladder carcinoma, transitional cell bladder carcinoma, gastric cancer, prostate adenocarcinoma, breast cancer, prostate cancer, lung cancer, colon carcinoma, Hodgkin's and non-Hodgkin's lymphoma, chronic lymphocytic leukemia (CLL), acute lymphoblastic leukemia (ALL), acute myeloblastic leukemia (AML), a solid tissue sarcoma, colon carcinoma, non-small cell lung carcinoma, squamous cell lung carcinoma, colorectal carcinoma, hepato-carcinoma, pancreatic cancer, and head and neck carcinoma.

46. The method of claim 42, wherein said cancer cell is a cell of a multiple myeloma.

47. The method according to any one of claims 41-46, wherein said method comprises inhibiting, delaying and/or preventing the growth of a tumor and/or spread of malignant tumor cells.

48. The method according to any one of claims 41-47, wherein said contacting comprises systemically administering said construct or formulation to a mammal.

49. The method according to any one of claims 41-47, wherein said contacting comprises administering said construct or formulation directly into a tumor site.

50. The method according to any one of claims 41-47, wherein said contacting comprises administering said construct or formulation via a route selected from the group consisting of oral administration, intravenous administration, intramuscular administration, direct tumor administration, inhalation, rectal administration, vaginal administration, transdermal administration, and subcutaneous depot administration.

51. The method according to any one of claims 41-47, wherein said contacting comprises administering said construct or formulation intravenously.

52. The method according to any one of claims 41-51, wherein said cell is a cell in a human.

53. The method according to any one of claims 41-51, wherein said cell is a cell in a non-human mammal.

54. The method of claim 41, wherein said cancer cell is a cell produced by a multiple myeloma.

55. The method according to any one of claims 41-54, wherein said method comprises co-administration of said chimeric construct with bortezomib.

56. The method according to any one of claims 41-55, wherein said method comprises co-administration of said chimeric construct with ibrutinib.

57. The method according to any one of claims 55-56, wherein said co-administration provides a synergistic effect.
